(19) United States
(12) Patent Application Publication
(10) Pub. No.: US 2018/0312584 A1
(43) Pub. Date: Nov. 1, 2018

(54) ANTI-IL-17A AND IL-17F CROSS REACTIVE ANTIBODY VARIANTS AND COMPOSITIONS COMPRISING AND METHODS OF MAKING AND USING SAME

(71) Applicant: Genentech, Inc., South San Francisco, CA (US)

(72) Inventors: GALAHAD DEPERALTA, S. SAN FRANCISCO, CA (US); AARON WECKSLER, EL GRANADA, CA (US); MELISSA ALVAREZ, S. SAN FRANCISCO, CA (US)

(73) Assignee: Genentech, Inc., South San Francisco, CA (US)

(21) Appl. No.: 15/835,811

(22) Filed: Dec. 8, 2017

Related U.S. Application Data
(63) Continuation of application No. 15/581,936, filed on Apr. 28, 2017.
Application No. 15/581,936 is a continuation of application No. PCT/US2015/058342, filed on Oct. 30, 2015.
(60) Provisional application No. 62/073,574, filed on Oct. 31, 2014.

(51) Int. Cl. C07K 16/24; C07K 16/06

(52) U.S. Cl. CPC C07K 16/244; C07K 2317/92; C07K 2317/90; C07K 2317/41; C07K 2317/33; C07K 16/065

(57) ABSTRACT

The present application relates to variants of an anti-IL-17A/F antibody, in particular, an glycosylation variant, a charge variant, an acidic variant, a HMWS variant, a reduction-resistant cross-linked variant, as well as compositions comprising the anti-IL-17A/F antibody and variant(s) thereof, methods of making and characterizing, and method of using the compositions thereof.

### CROSS REFERENCE

This application is a continuation of U.S. Ser. No. 15/581,936, filed Apr. 28, 2017, which is a continuation of International Application No. PCT/US2015/058342 having an international filing date of Oct. 30, 2015, the entire contents of which are incorporated herein by reference, and which claims the benefit of priority under 35 U.S.C. § 119 to U.S. Provisional Patent Application No. 62/073,574 filed Oct. 31, 2014, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 4, 2017, is named P32377US2SeqList.txt and is 13,823 bytes in size.

### FIELD OF THE INVENTION

The invention relates to variants of an anti-IL-17A and IL-17F cross-reactive antibody (anti-IL-17A/F antibody). In particular, the invention relates to glycosylation variants, acidic variants, charge variants, high-molecular-weight-species (HMWS) variants, and reduction-resistant (RR) cross-linked variants. The invention further relates to the isolated variants, compositions and pharmaceutical compositions comprising the antibody and the variants thereof, and articles of manufacture comprising the antibody and the variants, as well as methods of making, evaluating and characterizing the variants and compositions thereof.

### BACKGROUND OF THE INVENTION

Interleukin-17 (IL-17 or IL-17A) is a T-cell derived pro-inflammatory molecule that stimulates epithelial, endothelial and fibroblastic cells to produce other inflammatory cytokines and chemokines including IL-6, IL-8, G-CSF, and MCP-1. See, Yao, Z. et al., J. Immunol., 122(12):5483-5486 (1995); Yao, Z. et al, Immunity, 3(6):811-821 (1995); Fossiez, F., et al., J. Exp. Med., 183(6): 2593-2603 (1996); Kennedy, J., et al., J Interferon Cytokine Res., 16(8):611-7 (1996); Cai, X. Y., et al., Immunol. Lett, 62(1):51-8 (1998); Jovanovic, D. V., et al., J. Immunol., 160(7):3513-21 (1998); Laan, M., et al., J Immunol., 162(4):2347-52 (1999); Linden, A., et al., Eur Respir J, 15(5):973-7 (2000); and Aggarwal, S. and Gurney, A. L., J Leukoc Biol. 71(1):1-8 (2002)]. IL-17A also synergizes with other cytokines including TNF-α and IL-1β to further induce chemokine expression (Chabaud, M., et al., J. Immunol. 161(1):409-14 (1998)).

IL-17A has further been shown, by intracellular signaling, to stimulate Ca²⁺ influx and a reduction in [cAMP]ᵢ in human macrophages (Jovanovic et al, J Immunol., 160:3513 [1998]). Fibroblasts treated with IL-17 induce the activation of NF-κB, [Yao et al., Immunity, 3:811 (1995), Jovanovic et al., supra], while macrophages treated with it activate NF-κB and mitogen-activated protein kinases (Shalom-Barek et al, J. Biol. Chem., 273:27467 [1998]). Additionally, IL-17 also shares sequence similarity with mammalian cytokine-like factor 7 that is involved in bone and cartilage growth. Other proteins with which IL-17 polypeptides share sequence similarity are human embryo-derived interleukin-related factor (EDIRF) and interleukin-20.

Interleukin 17A is recognized as the prototype member of an emerging family of cytokines. The large scale sequencing of the human and other vertebrate genomes has revealed the presence of additional genes encoding proteins related to IL-17A, thus defining a new family of cytokines. There are at least 6 members of the IL-17 family in humans and mice including IL-17B, IL-17C, IL-17D, IL-17E and IL-17F. See WO 01/46420. The gene encoding human IL-17F is located adjacent to IL-17 (Hymowitz, S. G., et al., Embo J, 20(19):5332-41 (2001)). IL-17A and IL-17F share about 44% amino acid identity whereas the other members of the IL-17 family share a more limited 15-27% amino acid identity suggesting that IL-17A and IL-17F form a distinct subgroup within the IL-17 family (Starnes, T., et al., J Immunol. 167(8):4137-40 (2001); Aggarwal, S. and Gurney, A. L., J. Leukoc Biol, 71(1):1-8 (2002)). Each member of the IL-17 family forms homodimer. IL-17A and IL-17F additionally form IL-17AF heterodimer.

Human IL-17AF heterodimer is a distinctly new cytokine, distinguishable from human IL-17A and IL-17F in both protein structure and in cell-based activity assays. Through the use of purified recombinant human IL-17AF as a standard, a human IL-17AF-specific ELISA has been developed. Through the use of this specific ELISA, the induced expression of human IL-17AF was detected, confirming that IL-17AF heterodimer is naturally produced from activated human T cells in culture. Hence, IL-17AF is a distinctly new cytokine, detectable as a natural product of isolated activated human T cells, whose recombinant form has been characterized, in both protein structure and cell-based assays, as to be different and distinguishable from related cytokines. See, e.g., US20060270003 and WO2008/067223. Similar to IL-17A and IL-17F homodimer, IL-17AF heterodimer cytokine has been reported to signal through the IL-17RA/IL-17RC receptor complex (Wright et al., J Immunol 181(4):2799-805 (2008)). Antagonists to IL-17A and IL-17F, such as antibody antagonists (also referred to anti-IL-17A and IL-17F cross-reactive antibody or anti-IL-17A/F antibody), have been developed for treating IL-17A and IL-17F associated disorders (see e.g., U.S. Pat. No. 8,715,669 and U.S. Pat. No. 8,771,697, incorporated herein by reference).

### SUMMARY

The anti-IL-17A and IL-17F cross-reactive antibody comprises heavy chain variable domain CDR1 comprising the sequence of DYAMH (SEQ ID NO:1), CDR2 comprising the sequence of GINWSSGGIGYADSVKG (SEQ ID NO:2), CDR3 comprising the sequence of DIGGFGEFYWNFGL (SEQ ID NO:3), and light chain variable domain CDR1 comprising the sequence of RASQSVRSYLA (SEQ ID NO:4), CDR2 comprising the sequence of DASNRAT (SEQ ID NO:5), and CDR3 comprising the sequence of QQRSNWPPAT (SEQ ID NO:6). See U.S. Pat. No. 8,715,669, incorporated herein by reference in its entirety. The anti-IL-17A/F antibody binds to human IL-17AA homodimer, IL-17FF homodimer and IL-17AF heterodimer with high affinity and neutralizes human IL-17AA homodimer, IL-17FF homodimer and IL-17AF heterodimer-induced pro-inflammatory activities. Exemplary full-length human IL-17A and IL-17F amino acid sequences are shown in SEQ ID NO:12, and SEQ ID NO:13, respectively. Variants of the anti-IL-17A/F antibody described herein have not been previously reported.

The invention relates to variants of the anti-IL-17A/F antibody, in particular, a glycosylation variant, a HMWS variant, a reduction-resistant (RR) cross-linked variant, a charge variant, and an acidic variant.

Thus, in a first aspect, the invention provides compositions comprising an anti-IL-17A and anti-IL-17 F cross-reactive antibody comprising a heavy chain variable region CDR1 comprising the amino acid sequence of SEQ ID NO:1, CDR2 comprising the amino acid sequence of SEQ ID NO:2, CDR3 comprising the amino acid sequence of SEQ ID NO:3, and a light chain variable region CDR1 comprising the amino acid sequence of SEQ ID NO:4, CDR2 comprising the amino acid sequence of SEQ ID NO:5, and CDR3 comprising the amino acid sequence of SEQ ID NO:6, and a glycosylation variant thereof. In certain embodiments, the glycosylation is in the heavy chain variable region. In certain embodiments, the antibody or variant thereof is of the IgG class. In certain embodiments, the antibody or variant thereof is of the IgG1, IgG2, or IgG4 isotype. In certain embodiments, the antibody or a variant thereof is a monoclonal antibody, a fully human antibody, a humanized antibody, a chimeric antibody or a multi-specific antibody (e.g., a bispecific antibody). In certain other embodiments, the glycosylation variant is a heterodimer variant wherein only one half-antibody or only one heavy chain variable region is glycosylated. In certain embodiments, the glycosylation variant is a homodimer variant wherein both half-antibodies or both heavy chain variant regions are glycosylated. In certain embodiments, the glycosylation is in the heavy chain variable region CDR2, preferably at the Asn of SEQ ID NO:2. In certain embodiments, the heavy chain variable region comprises the amino acid sequence that has at least about 95%, at least about 96%, at least about 97%, at least about 98%, at least about 99%, or 100% sequence identity to the amino acid sequence of SEQ ID NO:7 and/or the light chain variable region comprises the amino acid sequence that has at least about 95%, at least about 96%, at least about 97%, at least about 98%, at least about 99%, or 100% sequence identity to the amino acid sequence of SEQ ID NO:8. In certain other embodiments, the antibody comprises a heavy chain comprising the amino acid sequence that has at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence of SEQ ID NO:9, and/or a light chain comprising the amino acid sequence that has at least about 85%, at least about 90%, at least about 91%, at least about 92%, at least about 93%, at least about 94%, at least about 95%, at least about 96%, at least about 97%, at least about 98%, at least about 99%, or 100% sequence identity to the amino acid sequence of SEQ ID NO:10. In certain embodiments, the antibody comprises a heavy chain comprising the amino acid sequence of SEQ ID NO:9 and a light chain comprising the amino acid sequence of SEQ ID NO:10. In certain embodiments, the anti-IL-17A/F antibody is referred to as the anti-IL17-A/F antibody MCAF5352A. In certain embodiments, the amount of the glycosylation variant in the composition is no more than 4%. In certain other embodiments, the amount of the glycosylation variant in the composition is no more than 2% of the glycosylation variant. In certain other embodiments, the glycosylated variant is detected, characterized and analyzed by size exclusion high performance liquid chromatography (SE-HPLC). In certain embodiments, the amount of the glycosylation variant in the composition is no more than 2% as measured by SE-HPLC. In certain embodiments, the amount of the glycosylation variant in the composition is more than 0%. In certain embodiments, the antibody is produced in mammalian cells, such as CHO cells.

In yet other embodiments, the composition comprises a glycosylation variant and further comprises one or more additional variants of the antibody, wherein the additional variants are selected from the group consisting of a high-molecular-weight-species (HMWS) variant, reduction-resistant (RR) cross-linked variant, and acidic variant. In certain embodiments, the antibody is produced in mammalian cells, such as CHO cells.

The anti-IL-17A/F antibody MCAF5253A exhibits atypical photo-sensitivity which was initially observed to lead to discoloration (yellowing) with an Aₘₐₓ˜420-440 nm, and was found to be associated with high molecular weight species (HMWS) formation. N₂-purging of anti-IL17 samples reduced both the discoloration and HMWS formation, suggesting oxidative-driven processes. Characterization of the photo-sensitivity of the antibody using various bioanalytical techniques including without limitation, charge variant analysis, SE-HPLC, capillary electrophoresis-sodium dodecyl sulfate (CE-SDS), tryptic peptide mapping, and intact/reduced mass analysis. A novel organic phase-size exclusion chromatography method, designated OP-SEC, was developed to enable fractionation and enrichment of putative non-reducible HMWS (NR-HMWS or RR-HMWS), which were demonstrated to be photo-induced scrambled disulfide bond cross-linked peptides. The cross-linked fraction consists of mainly heavy chain-heavy chain (HC-HC) (>90%) scrambled disulfides with a minor component of heavy chain-light chain (HC-LC) scrambled disulfides.

Thus, in certain embodiments, the composition further comprises a RR cross-linked variant. In certain other embodiments, the amount of an RR cross-linked variant in the composition is no more than about 1% to no more than about 3%. In certain other embodiments, the amount of the RR cross-linked variant in the composition is no more than about 3%. In certain embodiments, the amount of RR cross-linked variant in the composition is measured by reducing OP-SEC (organic phase size exclusion chromatography) or reducing CE-SDS (capillary electrophoresis-SDS) of the reduced antibody. In certain embodiments, the RR cross-linked variant in the composition is no more than about 1% as determined by reducing CE-SEC. In certain embodiments, the amount of the RR cross-linked variant in the composition is no more than about 3% as determined by OP-SEC of the reduced antibody. In certain embodiments, the RR cross-linked variant comprises a cross link between Cys and Cys. In certain other embodiments, the RR cross-linked variant comprises a cross link between Trp and Trp. In certain embodiments, the cross link is an intermolecular or intramolecular cross link. In certain embodiments, the RR cross-linked variant comprises a heavy chain-heavy chain cross-link. In certain other embodiments, RR cross-linked variant comprises a heavy chain-light chain cross-link. In certain other embodiments, the RR cross-linked variant is induced by light, for example ambient light.

In certain other embodiments, the composition further comprises a HMWS variant. In certain other embodiments, the amount of the HMWS variant in the composition is no more than about 1%. In certain other embodiments, the amount of the HMWS variant is determined by SE-HPLC. In certain particular embodiments, the amount of the HMWS variant in the composition is no more than about 1% as determined by SE-HPLC.

In certain embodiments, the composition further comprises an acidic variant. In certain particular embodiments, the amount of the acidic variant in the composition is no more than about 42%. In certain embodiments, the amount of the acidic variant is determined by imaged capillary isoelectric-focusing (icIEF). In certain embodiments, the amount of the acidic variant in the composition is no more than about 42% as determined by icIEF.

In a further aspect, the invention provides compositions comprising an anti-IL-17A and anti-IL-17 F cross reactive antibody comprising a heavy chain variable region CDR1 having the amino acid sequence of SEQ ID NO: 1, CDR2 having the amino acid sequence of SEQ ID NO:2, CDR3 having the amino acid sequence of SEQ ID NO:3, and a light chain variable region CDR1 having the amino acid sequence of SEQ ID NO:4, CDR2 having the amino acid sequence of SEQ ID NO:5, and CDR3 having the amino acid sequence of SEQ ID NO:6, wherein the composition comprises one or more of a glycosylation variant, an RR cross-linked variant, a HMWS variant, and an acidic variant.

In certain embodiments, the composition comprises an RR cross-linked variant. In certain embodiments, the amount of the RR cross-linked variant in the composition is no more than about 3% as determined by reducing OP-SEC. In certain other embodiments, the composition comprises a HMWS variant. In certain other embodiments, the amount of the HMWS variant in the composition is no more than about 1% as determined by SE-HPLC. In certain other embodiments, the composition comprises an acidic variant. In certain other embodiments, the amount of the acidic variant in the composition is no more than about 42% as determined by imaged capillary isoelectric-focusing (icIEF). In certain embodiments, the composition comprises a glycosylation variant, a HMWS variant, an RR cross-linked variant, and an acidic variant, in which the amount of the glycosylation variant in the composition is no more than about 2%, the amount of the RR cross-linked variant in the composition is no more than about 3%, the amount of the HMWS variant in the composition is no more than about 1%, and the amount of the acidic variant in the composition is no more than about 42%.

In a further aspect, the invention provides pharmaceutical compositions comprising the composition described herein and one or more pharmaceutically acceptable excipients. In certain embodiments, the pharmaceutical compositions comprise the main species of the anti-IL-17A/F antibody and a variant thereof.

In yet another aspect, the invention provides an article of manufacturing comprising a container with the pharmaceutical composition described herein and a package insert with prescribing information instructing the use thereof to use the pharmaceutical composition to treat a patient in need thereof.

In a further aspect, the invention provides methods of making the compositions described herein, comprising producing a composition comprising the main species of the IL-17A/F antibody and one or more variants thereof; subjecting the composition produced to one or more analytical assays to evaluate the amount of the one or more variants in the composition. In certain embodiments, the method further comprises subjecting the composition produced to one or more rounds of purification.

In yet another aspect, the invention provides methods of treating an immune-related disease or disorder, such as an autoimmune disease or disorder and an inflammatory disease or disorder, or a cell proliferation-related disease or disorder comprising administering to a subject in need thereof the pharmaceutical composition described herein. In certain embodiments, the immune-related diseases or disorders include without limitation systemic lupus erythematosis, rheumatoid arthritis, psoriatic arthritis, osteoarthritis, juvenile chronic arthritis, spondyloarthropathies, systemic sclerosis, idiopathic inflammatory myopathies, Sjögren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious, autoimmune chronic active hepatitis, primary biliary cirrhosis, granulomatous hepatitis, and sclerosing cholangitis, inflammatory bowel disease, ulcerative colitis, Crohn's disease, gluten-sensitive enteropathy, and Whipple's disease, bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, transplantation associated diseases including graft rejection and graft-versus-host-disease. In certain other embodiments, the immune-related disorder is asthma, multiple sclerosis, rheumatoid arthritis, inflammatory bowel disease, ulcerative colitis, lupus erythematosus, psoriasis, chronic obstructive pulmonary disease, or idiopathic pulmonary fibrosis.

In certain other embodiments, the cell proliferation-related disorder is, without limitation, colorectal cancer, renal cell cancer (e.g., renal cell carcinoma), melanoma, bladder cancer, ovarian cancer, breast cancer (e.g., triple-negative breast cancer, HER2-positive breast cancer, or hormone receptor-positive cancer), and non-small-cell lung cancer (e.g., squamous non-small-cell lung cancer or non-squamous non-small-cell lung cancer). In some embodiments, a cancer to be treated by the methods of the present disclosure includes, but is not limited to, a carcinoma, lymphoma, blastoma, sarcoma, and leukemia. In some embodiments, a cancer to be treated by the methods of the present disclosure includes, but is not limited to, squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL;

high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In some embodiments, the cancer may be an early stage cancer or a late stage cancer. In some embodiments, the cancer may be a primary tumor. In some embodiments, the cancer may be a metastatic tumor at a second site derived from any of the above types of cancer.

Any and every embodiment described above applies to any and every aspect of the invention, unless the context clearly indicates otherwise. All embodiments within and between different aspects can be combined unless the context clearly dictates otherwise.

Specific embodiments of the present invention will become evident from the following more detailed description of certain preferred embodiments and the claims.

### BRIEF DESCRIPTION OF THE DRAWINGS

FIG. 1A: SEC chromatogram of a composition of anti-IL-17A/F antibody MCAF5352A showing 2% of Peak 1 in the composition. FIG. 1B shows an expanded view.

FIG. 2: SEC chromatogram of enriched Peak 1 after trypsin and PNGase digestion.

FIGS. 3 A and 3B: LC-MS analysis of PNGase treated enriched Peak 1. FIG. 3A: the MS-TIC results show that removal of the glycans of enriched Peak 1 produced a new peak in the chromatogram. FIG. 3B, top panel—the raw MS data showed the mass of unmodified, non-glycosylated parent tryptic peptide HC44-65; the bottom panel showed the deglycosylated HC 44-65, where Asn52 was replaced by Asp52. The deglycosylated peptide has a different retention time from the non-glycosylated HC 44-65 peptide and has a slightly higher mass, as expected. The presence of Asp52 was confirmed by N-terminal sequencing of the collected peptide.

FIGS. 4A and 4B: Photo-induced discoloration of the anti-IL-17A/F antibody MCAF5352A. FIG. 4A, Photographic image of observable discoloration at 0 hours, 6 hours and 24 hours light exposure under the ICH guidelines. FIG. 4B UV-Visible spectroscopic profile of 24 hours light exposed MCAF5352A. Unique UV absorption profile with Absₘₐₓ˜430 nm observed in light-exposed anti-IL17A/F samples. Similar color changes are observed with ambient light exposure, though at longer exposure times (data not shown).

FIGS. 5A and 5B: Photo-induced high molecular weight species formation (HMWS) observed by SEC. FIG. 5A, intact size exclusion chromatography (SEC) analysis of light-exposed anti-IL-17A/F antibody. FIG. 5B, Quantitative analysis demonstrated linear correlation between HMWS formation and light exposure.

FIGS. 6A and 6B: Use of novel organic phase-size exclusive chromatograph (OP-SEC) to identify reduction-resistant cross-linked species. FIG. 6A: OP-SEC analysis of light-exposed anti-IL17A/F MCAF5352A treated with DTT. FIG. 6B: Quantitative analysis demonstrated linear correlation between reduction-resistant cross-linked species formation and light exposure. Cross-linked species were then enriched by fraction-collection.

FIGS. 7A and 7B: Photo-induced increase in charge variants of MCAF5352A. FIG. 7A: Charge variant detected using icIEF analysis upon light exposure. FIG. 7B: Quantitative analysis demonstrated increases in acidic charge variants.

FIGS. 8A and 8B: Evidence for both intra-molecular and inter-molecular cross-linking. FIG. 8A, OP-SEC analysis of reduced anti-IL-17A/F antibody MCAF5352 sample. FIG. 8B, Quantitative analysis demonstrated NR (non-reducible, or RR, reduction-resistant)-HMWS in both the SEC main peak and HMWS fractions, indicating both intra-molecular and inter-molecular cross-linking, respectively.

FIG. 9A-C: Site-specific photo-induced oxidation analyzed by tryptic mapping. FIG. 9A, Quantitative analysis of methionine oxidation; FIG. 9B, Quantitative analysis of most susceptible tryptophan oxidation; FIG. 9C, Quantitative analysis of overall tryptophan oxidized species.

FIG. 10: Direct correlation between the levels of site-specific oxidation, HMWS as determined by SEC, and RR cross-linked species as determined by OP-SEC.

FIG. 11A-C: ESI-TOF-MS analysis demonstrated cross-linked species composition. FIG. 11A: Full MS of fractionated/enriched cross-linked species from reducing OP-SEC. FIG. 11B: Expanded MS showing Heavy chain-Heavy chain (HC-HC) putative cross-linked species, and FIG. 11C: Expanded MS showing Heavy chain-Light chain (HC-LC) putative cross-linked species.

FIGS. 12A and 12B: N₂-purging reduced discoloration, HMWS and cross-linked species formation, indicating that discoloration, HMWS formation and RR cross linking involved oxidative processes. FIG. 12A, SEC analysis of N₂-purged sample, where a decrease in discoloration with N₂-purging was observed (inset); FIG. 12B, reducing OP-SEC analysis of N₂-purged sample.

FIGS. 13A and 13B: N₂-purging reduced global oxidation. FIG. 13A, RP-HPLC analysis of global oxidation of the Fc, LC (light chain) and Fab regions of the anti-IL-17A/F Ab MCAF5352A; FIG. 13B, Quantitative analysis indicated significant reduction in Fc-oxidation from N₂-purging.

FIGS. 14A and 14B: NaN₃-treatment suggests singlet oxygen-drive processes. FIG. 14A shows a direct correlation between discoloration and NaN₃-treatment concentrations. FIG. 14B, Protective effects of NaN₃-treatment on HMWS formation measured by SEC and cross-link formation as measured by OP-SEC.

FIGS. 15A-1 and 15A-2: Identification of RR cross-linked peptide in light-exposed IL-17A/F antibody MCAF5352A using O¹⁸-labeling workflow and MS/MS. FIG. 15A-1 and FIG. 15A-2, hinge-Fc RR cross-linked disulfide bond between C232 (hinge) and C373 (Fc) (SEQ ID NOS 14 and 15, respectively, in order of appearance); FIG. 15B-1 and FIG. 15B-2, hinge-Fab RR cross-linked disulfide bond between C235 (hinge) and C96 (Fab) (SEQ ID NOS 16 and 15, respectively, in order of appearance).

FIGS. 16A and 16B: The illustration in FIG. 16A indicates known disulfide bonds in a full-length antibody. FIG. 16B lists light-induced reduction resistant scrambled disulfide bonds identified in the subject antibody by database search (Mass Matrix Software Suite). Two scrambled disulfide bonds were confirmed and the others were detected positive with O¹⁸-labeling, suggesting their presence in the light-induced RR cross-linked variant. Several of these scrambled disulfides involve the hinge region. FIG. 16B.

### DETAILED DESCRIPTION OF THE PREFERRED EMBODIMENTS

All publications, patents and patent applications cited herein are hereby expressly incorporated by reference for all purposes.

### I. Definitions

As used herein, the singular forms “a”, “an” and “the” include plural referents unless the context clearly dictates otherwise.

A “glycosylation variant” of an antibody as used herein is an antibody with one or more carbohydrate moieties attached to the variable region of the antibody as compared to the main species of the antibody that is not glycosylated in the variable region. In one embodiment, the glycosylation variant has oligosaccharide structures attached to one or both heavy chains of the antibody. In certain embodiments, the glycosylation site is at the Asn of amino acid residue of the heavy chain variable region (VH) CDR2 (SEQ ID NO:2), or amino acid residue 52 of VH. In certain embodiments, both heavy chain variable regions are glycosylated (homodimer variant). In certain other embodiments, only one of the two heavy chain variable regions is glycosylated (heterodimer variant). In certain embodiments, the oligosaccharides covalently attached to the Asn in the heavy chain variable region can be heterogeneous among the glycosylation variants.

The term “anti-IL-17A and anti-IL-17F cross reactive antibody,” “anti-IL-17A/F antibody” or “anti-IL-17A/F cross-reactive antibody” refers to an antibody that binds to and neutralizes IL-17A homodimer, IL-17F homodimer and IL-17AF heterodimer.

The term “main species antibody” or “wild type antibody” herein refers to the antibody amino acid sequence structure in a composition which is the quantitatively predominant antibody molecule in the composition. Preferably, the main species antibody is an anti-IL-17A/F antibody, such as an antibody that binds to and neutralizes IL-17A homodimer, IL-17F homodimer and IL-17AF heterodimer. In one embodiment, the main species antibody is one comprising CDR-H1 (SEQ ID NO: 1), CDR-H2 (SEQ ID NO: 2), and CDR-H3 (SEQ ID NO: 3), CDR-L1 (SEQ ID NO: 4), CDR-L2 (SEQ ID NO: 5) and CDR-L3 (SEQ ID NO: 6). In certain embodiments, the anti-IL-17A/F antibody comprises a heavy chain variable region comprising the sequence of SEQ ID NO:7, and/or a light chain variable region comprising the sequence of SEQ ID NO:8. In one embodiment, the main species antibody is MCAF5352A.

An “intact antibody” herein is one which comprises two antigen binding regions, and an Fc region. In certain embodiments, the intact antibody has a functional Fc region. In one embodiment, “intact IL-17A/F antibody MCAF5352A” has a molecular weight of about 148,724 Da as measured by LC/MS including the Fc glycan without the C-terminal Lys.

A “low-molecular-weight-species” or “LMWS” variant of the anti-IL-17A/F antibody comprises a fragment of the antibody that has a molecular weight less than that of the main species or intact anti-IL-17A/F antibody. In certain embodiments, the LMWS variant of the anti-IL-17A/F antibody MCAF5352A comprises a fragment of the antibody that has a molecular weight less than that of the main species or intact anti-IL-17A/F antibody MCAF5352A. The LMWS can be detected by size exclusion high performance liquid chromatography (SE-HPLC) and/or non-reduced Capillary Electrophoresis with Sodium Dodecyl Sulfate (CE-SDS).

A “high-molecular-weight-species” or “HMWS” variant comprises a preparation of the anti-IL-17A/F antibody having a molecular weight that is greater than the main species or intact anti-IL-17A/F antibody. In certain embodiments, the HMWS variant comprises a preparation of the anti-IL-17A/F antibody MCAF5352A having a molecular weight that is greater than the intact or main species anti-IL-17A/F antibody MCAF5352A (e.g. where the intact anti-IL-17A/F antibody MCAF5352A has a molecular weight of about 148,724 Da). The HMWS can be detected by size exclusion high performance liquid chromatography (SE-HPLC) and/or non-reduced Capillary Electrophoresis with Sodium Dodecyl Sulfate (CE-SDS). In certain embodiments, the HMWS is light-induced HMW S.

An amino acid sequence variant antibody is an antibody with an amino acid sequence which differs from a main species antibody. Ordinarily, amino acid sequence variants will possess at least about 70% homology with the main species antibody, and preferably, they will be at least about 80%, and more preferably at least about 90% homologous with the main species antibody. In certain embodiments, amino acid sequence variants will possess at least about 70%, about 80%, about 85%, about 90%, about 92%, about 95%, about 98%, about 99% sequence identity to the main species antibody. The amino acid sequence variants possess substitutions, deletions, and/or additions at certain positions within or adjacent to the amino acid sequence of the main species antibody. Examples of amino acid sequence variants herein include deamidated antibody variant, antibody with an amino-terminal leader extension (e.g. VHS-) on one or two light chains thereof, antibody with a C-terminal lysine residue on one or two heavy chains thereof, etc., and includes combinations of variations to the amino acid sequences of heavy and/or light chains.

In certain embodiments, antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

A “deamidated” antibody is one in which one or more asparagine residues thereof has been derivatized, e.g. to an aspartic acid, a succinimide, or an iso-aspartic acid.

An “acidic variant” is a variant of the main species antibody which is more acidic than the main species antibody. An acidic variant has gained negative charge or lost positive charge relative to the main species antibody. In certain embodiments, the acidic variant may occur as a result of methionine or tryptophan oxidation. Such acidic variants can be resolved using a separation methodology, such as ion exchange chromatography, that separates proteins according to charge. Acidic variants of a main species antibody elute earlier than the main peak upon separation by cation exchange chromatography.

A “charge variant” refers to a variant that carries a different total charge than the main species antibody at a given pH. A charge variant can be an acidic variant (variant that has gained negative charge or lost positive charge) or a basic variant (variant that has gained positive charge or lost negative charge). In one embodiment, modification(s) on one or more amino acid residues of the antibody results in different total charge of the charge variant as compared to the main species antibody.

A “reduction resistant (RR) cross-linked variant” refers to a variant of the main species antibody that cannot be chemically reduced to a heavy chain and a light chain by a reducing agent such as dithiothreitol. A RR cross-linked variant is also referred to as a non-reducible (NR) cross-linked variant. Such variants can be assessed by treating the composition with a reducing agent and evaluating the resulting composition using a methodology that evaluates protein size, such as Capillary Electrophoresis with Sodium Dodecyl Sulfate (CE-SDS), or organic phase size exclusion chromatography (OP-SEC) as described herein. In certain embodiments, the RR cross-linked variant results from exposure to light, for example, ambient light. In certain other embodiments, the RR cross-link occurs between Cys and Cys residues or Trp and Trp residues. In certain embodiments, the RR cross-link occurs intermolecularly, e.g., between one antibody molecule and another antibody molecule; in certain other embodiments, the RR cross-link occurs intramolecularly, e.g., within one full length antibody molecule.

A “C-terminal lysine variant” refers to a variant comprising a lysine (K) residue at the C-terminus of the heavy chain thereof.

A “methionine-oxidized variant” refers to a variant comprising one or more oxidized methionine residues therein, e.g. oxidized Met258, Met364, and/or Met434 according to the full-length heavy chain comprising the sequence of SEQ ID NO:9.

A “tryptophan-oxidized variant” refers to a variant comprising one or more oxidized tryptophan residues therein. In certain embodiments, the tryptophan-oxidized variant comprises oxidation of one or more tryptophan residues selected from W53, W108 of the heavy chain variable region according to the VH sequence comprising the sequence of SEQ ID NO:7, and W94 of the light chain variable region according to the VL sequence comprising the sequence of SEQ ID NO:8.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. In certain embodiments, the composition comprises an IL-17A/F antibody that is a fully human antibody, a humanized antibody, or a chimeric antibody. In certain other embodiments, the antibody is a bispecific or multispecific antibody. In certain embodiments, the bispecific or multispecific antibody has at least two different binding specificities, one of the binding specificities being for the IL-17A homodimer, IL-17F homodimer and IL-17AF heterodimer.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgAᵢ, and IgA₂. The heavy chain constant domains that correspond to the different classes of immunoglobulins are called α, δ, ε, γ, and μ, respectively.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain (in the example of the heavy chain comprising the amino acid sequence of SEQ ID NO:9, Cys232 and Pro236, respectively). However, the C-terminal lysine of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 449 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K449 residues removed, antibody populations with no K449 residues removed, and antibody populations having a mixture of antibodies with and without the K449 residue.

Unless otherwise indicated, HVR residues and other residues in the variable domain (e.g., FR residues) are numbered herein according to Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc. Such effector functions generally require the Fc region to be combined with a binding domain (e.g. an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “naked antibody” is an antibody that is not conjugated to a heterologous molecule, such as a cytotoxic moiety or radiolabel.

An “immunoconjugate” is an antibody conjugated to one or more heterologous molecule(s), including but not limited to a cytotoxic agent.

An “analytical assay” is an assay which qualitatively assesses and/or quantitatively measures the presence or amount of an analyte (e.g. an antibody variant) in a composition. The composition subjected to the assay can be a purified composition, including a pharmaceutical composition.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies compositions comprising the main species antibody and a variant thereof of the invention are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result.

A “container” refers to an object that can be used to hold or contain a pharmaceutical composition or composition. Examples of containers herein include a vial, syringe, intravenous bag, etc.

An “intravenous bag” or “IV bag” is a bag that can hold a solution which can be administered via the vein of a patient. In one embodiment, the solution is a saline solution (e.g. about 0.9% or about 0.45% NaCl). Optionally, the IV bag is formed from polyolefin or polyvinal chloride.

A “vial” is a container suitable for holding a liquid or lyophilized preparation. In one embodiment, the vial is a single-use vial, e.g. a 20-cc single-use vial with a stopper.

A “package insert” is a leaflet that, by order of the Food and Drug Administration (FDA) or other regulatory authority, must be placed inside the package of every prescription drug. The leaflet generally includes the trademark for the drug, its generic name, and its mechanism of action; states its indications, contraindications, warnings, precautions, adverse effects, and dosage forms; and includes instructions for the recommended dose, time, and route of administration.

A “pharmaceutical composition” is a composition comprising a pharmaceutically active drug (for example, the anti-IL-17A/F antibody MCAF5352A and variant forms thereof such as those disclosed herein) and one or more “pharmaceutically active excipients” (e.g. buffer, stabilizer, tonicity modifier, preservative, surfactant, etc.) that can be safely administered to a human patient. Such compositions may be liquid or lyophilized, for example. In certain embodiments, the composition further comprises one or more additional active drugs.

The term “immune related disease” means a disease in which a component of the immune system of a mammal causes, mediates or otherwise contributes to a morbidity in the mammal. Also included are diseases in which stimulation or intervention of the immune response has an ameliorative effect on progression of the disease. Included within this term are immune-mediated inflammatory diseases, non-immune-mediated inflammatory diseases, infectious diseases, immunodeficiency diseases, neoplasia, etc.

The term “T cell mediated disease” means a disease in which T cells directly or indirectly mediate or otherwise contribute to morbidity in a mammal. The T cell mediated disease may be associated with cell mediated effects, lymphokine mediated effects, etc., and even effects associated with B cells if the B cells are stimulated, for example, by the lymphokines secreted by T cells.

Examples of immune-related and inflammatory diseases, some of which are immune or T cell mediated, which can be treated according to the invention include systemic lupus erythematosis, rheumatoid arthritis, juvenile chronic arthritis, spondyloarthropathies, systemic sclerosis (scieroderma), idiopathic inflammatory myopathies (dermatomyositis, polymyositis), Sjogren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barre syndrome, and chronic inflammatory demyelinating polyneuropathy, hepatobiliary diseases such as infectious hepatitis (hepatitis A, B, C, D, E and other non-hepatotropic viruses), asthma, autoimmune chronic active hepatitis, primary biliary cirrhosis, granulomatous hepatitis, and sclerosing cholangitis, inflammatory bowel disease (IBD), including ulcerative colitis, Crohn's disease, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, transplantation associated diseases including graft rejection and graft-versus-host-disease. Infectious diseases including viral diseases such as AIDS (HIV infection), hepatitis A, B, C, D, and E, herpes, etc., bacterial infections, fungal infections, protozoal infections and parasitic infections.

The term “cell proliferation-related disorder” or “cell proliferative disorder” or “proliferative disorder” refers to disorders that are associated with some degree of abnormal cell proliferation. In certain embodiments, the cell proliferative disorder is cancer. In some embodiments, the cell proliferative disorder is a tumor.

“Tumor,” as used herein, refers to all neoplastic cell growth and proliferation, whether malignant or benign, and all pre-cancerous and cancerous cells and tissues. The terms “cancer”, “cancerous”, “cell proliferative disorder”, “proliferative disorder” and “tumor” are not mutually exclusive as referred to herein.

In some embodiments, a cancer to be treated by the methods of the present disclosure includes, but is not limited to, colorectal cancer, renal cell cancer (e.g., renal cell carcinoma), melanoma, bladder cancer, ovarian cancer, breast cancer (e.g., triple-negative breast cancer, HER2-positive breast cancer, or hormone receptor-positive cancer), and non-small-cell lung cancer (e.g., squamous non-small-cell lung cancer or non-squamous non-small-cell lung cancer). In some embodiments, a cancer to be treated by the methods of the present disclosure includes, but is not limited to, a carcinoma, lymphoma, blastoma, sarcoma, and leukemia. In some embodiments, a cancer to be treated by the methods of the present disclosure includes, but is not limited to, squamous cell cancer, lung cancer (including small-cell lung cancer, non-small cell lung cancer, adenocarcinoma of the lung, and squamous carcinoma of the lung), melanoma, renal cell carcinoma, cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In some embodiments, the cancer may be an early stage cancer or a late stage cancer. In some embodiments, the cancer may be a primary tumor. In some embodiments, the cancer may be a metastatic tumor at a second site derived from any of the above types of cancer.

A “recombinant” protein is one which has been produced by a genetically modified host cell, such as a Chinese Hamster Ovary (CHO) host cell.

“Manufacturing scale” refers to production of a protein drug (e.g. antibody) at a commercial scale, e.g. at 12,000 liter (L) or more, using a commercial process approved by the FDA or other regulatory authority.

“Purifying” refers to one or more purification steps, such as Protein A chromatography, ion exchange chromatography, size exclusion chromatography, hydrophobic interaction column chromatography, etc.

“Isolated” variant refers to the variant which has been separated from the main species or wild-type antibody by one or more purification or analytical procedures. Such isolated variant can be evaluated for its biological activity and/or potency.

### II. Antibody Compositions

(a) Main Species Antibody

The antibody compositions herein comprise an antibody that binds human IL-17A, IL-17F and IL-17AF heterodimer (an anti-IL-17A/F antibody). In certain embodiments, the antibody is a human antibody. In certain other embodiments, the antibody is a humanized antibody. The humanized antibody may, for example, comprise hypervariable region derived from non-human source, which is incorporated into a human variable heavy domain. Unless specified, the variable domain numbering follows the numbering system set forth in Kabat et al., Sequences of Proteins of Immunological Interest, 5th Ed. Public Health Service, National Institutes of Health, Bethesda, Md. (1991).

In certain embodiments, the anti-IL-17A/F antibody comprises CDR-H1 (SEQ ID NO: 1), CDR-H2 (SEQ ID NO: 2), and CDR-H3 (SEQ ID NO: 3), CDR-L1 (SEQ ID NO: 4), CDR-L2 (SEQ ID NO: 5) and CDR-L3 (SEQ ID NO: 6). The invention also contemplates amino acid modifications of those CDR residues, e.g. where the modifications essentially maintain or improve affinity of the antibody. For example, an antibody variant for use in the methods of the present invention may have from about one to about seven or about five amino acid substitutions in the above variable heavy CDR sequences. Such antibody variants may be prepared by affinity maturation. Various forms of the humanized antibody or affinity matured antibody are contemplated. Alternatively, the humanized antibody or affinity matured antibody may be an intact antibody, such as an intact IgG1 antibody.

In certain embodiments, the anti-IL-17A/F antibody comprises a heavy chain variable region comprising the sequence of SEQ ID NO:7, and/or a light chain variable region comprising the sequence of SEQ ID NO:8. In certain particular embodiments, the anti-IL-17AF antibody comprises a heavy chain comprising the sequence of SEQ ID NO:9 and/or a light chain comprises the sequence of SEQ ID NO:10. In certain embodiments, the C-terminal Lys is optionally present in the heavy chain.

(VH)
SEQ ID NO: 7
EVQLVESGGG LVQPGRSLRL SCAASGFTFD DYAMHWVRQA
PGKGLEWVSG INWSSGGIGY ADSVKGRFTI SRDNAKNSLY
LQMNSLRAED TALYYCARDI GGFGEFYWNF GLWGRGTLVT
VSS
(VL)
SEQ ID NO: 8
EIVLTQSPAT LSLSPGERAT LSCRASQSVR SYLAWYQQKP
GQAPRLLIYD ASNRATGIPA RFSGSGSGTD FTLTISSLEP
EDFAVYYCQQ RSNWPPATFG GGTKVEIK
(HC)
SEQ ID NO: 9
EVQLVESGGG LVQPGRSLRL SCAASGFTFD DYAMHWVRQA
PGKGLEWVSG INWSSGGIGY ADSVKGRFTI SRDNAKNSLY
LQMNSLRAED TALYYCARDI GGFGEFYWNF GLWGRGTLVT
VSSASTKGPS VFPLAPSSKS TSGGTAALGC LVKDYFPEPV
TVSWNSGALT SGVHTFPAVL QSSGLYSLSS VVTVPSSSLG
TQTYICNVNH KPSNTKVDKR VEPKSCDKTH TCPPCPAPEL
LGGPSVFLFP PKPKDTLMIS RTPEVTCVVV DVSHEDPEVK
FNWYVDGVEV HNAKTKPREE QYNSTYRVVS VLTVLHQDWL
NGKEYKCKVS NKALPAPIEK TISKAKGQPR EPQVYTLPPS
REEMTKNQVS LTCLVKGFYP SDIAVEWESN GQPENNYKTT
PPVLDSDGSF FLYSKLTVDK SRWQQGNVFS CSVMHEALHN
HYTQKSLSLS PG
(LC)
SEQ ID NO: 10
EIVLTQSPAT LSLSPGERAT LSCRASQSVR SYLAWYQQKP
GQAPRLLIYD ASNRATGIPA RFSGSGSGTD FTLTISSLEP
EDFAVYYCQQ RSNWPPATFG GGTKVEIKRT VAAPSVFIFP
PSDEQLKSGT ASVVCLLNNF YPREAKVQWK VDNALQSGNS
QESVTEQDSK DSTYSLSSTL TLSKADYEKH KVYACEVTHQ
GLSSPVTKSF NRGEC
(full-length human IL-17A with the leader
sequence, Swiss-Prot Accession No. Q16552.1)
SEQ ID NO: 12
MTPGKTSLVS LLLLLSLEAI VKAGITIPRN PGCPNSEDKN
FPRTVMVNLN IHNRNTNTNP KRSSDYYNRS TSPWNLHRNE
DPERYPSVIW EAKCRHLGCI NADGNVDYHM NSVPIQQEIL
VLRREPPHCP NSFRLEKILV SVGCTCVTPI VHHVA
(full-length human IL-17F with the leader
sequence, Swiss-Prot Accession No. Q96PD4.3)
SEQ ID NO: 13
MTVKTLHGPA MVKYLLLSIL GLAFLSEAAA RKIPKVGHTF
FQKPESCPPV PGGSMKLDIG IINENQRVSM SRNIESRSTS
PWNYTVTWDP NRYPSEVVQA QCRNLGCINA QGKEDISMNS
VPIQQETLVV RRKHQGCSVS FQLEKVLVTV GCTCVTPVIH HVQ

(b) Glycosylation Variant

In one aspect, the invention provides a glycosylation variant antibody either in isolated form, enriched form or in a composition comprising the glycosylation variant and the main species antibody. In certain embodiments, the glycosylation is N-linked glycosylation on the Asn residue of CDR-H2 (SEQ ID NO:2). In certain embodiments, the amount of the glycosylation variant in the composition is no more than (i.e., equal or less than) about 10%, no more than about 9%, no more than about 8%, no more than about 7%, no more than about 6%, no more than about 5%, no more than about 4%, no more than about 3%, no more than about 2%, or no more than about 1%. In certain embodiments, the amount of the glycosylation variant in the composition is no more than about 2%. In certain embodiments, the amount of glycosylation in the composition is determined by LC-MS. An anti-IL-17A/F antibody composition containing high level of the glycosylation variant exhibits reduced binding, and/or reduced neutralizing activity to IL-17A, IL-17F and/or IL-17AF, and/or increased immunogenicity, and/or increased serum clearance.

(c) LMWS and HMWS Variants

The invention provides a low-molecule-weight species (LMWS) variant and/or a high-molecule-weight species (HMWS) variant of the anti-IL17A/F antibody either in an isolated form, an enriched form, or in a composition comprising the LMWS and/or HMWS variant and the main species antibody. The LMWS and HMWS variants can be isolated, characterized, and quantified using various techniques, including, without limitation, size exclusion high performance liquid chromatography (SE-HPLC), and/or Capillary Electrophoresis Sodium Dodecyl Sulfate (CE-SDS).

Using an SE-HPLC assay (e.g. as in Example 2), the amount of main species anti-IL-17A/F antibody and a HMWS variant or a LMWS variant in a composition may be:

Main Peak: ≥ about 98.9%, e.g., ≥ about 99.1%, ≥ about 94.9%, e.g., ≥ about 95.0%.
HMWS: ≤ about 1%, e.g., ≤ about 0.8%, ≤ about 4.9%, e.g. ≤ about 4.6%.
LMWS: ≤ about 0.5%, e.g., ≤ about 0.3%, ≤ about 0.2%, e.g. ≤ about 0.1%.

In certain embodiments, the amount of a HMWS variant in a composition is no more than (or equal or less than) about 10%, no more than about 9%, no more than about 8%, no more than about 7%, no more than about 6%, no more than about 5%, no more than about 4%, no more than about 3%, no more than about 2%, or no more than about 1%. In certain embodiments, the amount of a LMWS variant in a composition is no more than (or equal or less than) about 2%, no more than about 1%, no more than about 0.5%, no more than about 0.3%, or no more than about 0.1%. In certain embodiments, the amount of the HMWS variant or the LMWS variant in the composition is measured by SEC (or SE-HPLC). In certain embodiments, the composition comprises no more than about 1% of the HMWS variant and/or no more than about 0.1% of the LMWS variant, as measured by SEC. In certain embodiments, the HMWS variant is induced by light exposure. An anti-IL-17A/F antibody composition containing high level of HMWS variant exhibits reduced binding and/or neutralizing activity to IL-17A, IL-17F and/or IL-17AF, and/or increased immunogenicity, and/or increased serum clearance.

(d) RR Cross-Linked Variant

The invention relates to a reduction-resistant (RR) cross-linked variant of the anti-IL-17A/F antibody either in an isolated form, an enriched form, or in a composition comprising the RR cross-linked variant and the main species antibody. The RR cross-linked variant can be isolated, characterized, and quantified using various techniques, including, without limitation, size exclusion high performance liquid chromatography (SE-HPLC), organic phase SEC (OP-SEC) and/or Capillary Electrophoresis Sodium Dodecyl Sulfate (CE-SDS). OP-SEC may also be referred to reducing OP-SEC when the sample has been treated with a reducing agent such as DTT. In certain embodiments, the cross-link is induced by light exposure. In certain embodiments, the cross-link is between Cys and Cys residues. In certain other embodiments, the cross-link is between Trp and Trp residues. The cross-links can be intermolecular and intramolecular cross linking.

In certain embodiments, the amount of RR cross linked variant in the composition is no more than, i.e., equal or less than, about 6%, no more than about 5%, no more than about 4%, no more than about 3%, no more than about 2% or no more than about 1%. In certain embodiments, the amount of an RR cross-linked variant in the composition is determined by reducing OP-SEC. In certain embodiments, the amount of RR cross linked variant in the composition is no more than about 3% as determined by reducing OP-SEC. In certain embodiments, an anti-IL17A/F antibody composition containing high level of RR cross-linked variant exhibits reduced binding and/or reduced neutralizing activity to IL17A, IL17F and/or IL17AF, and/or increased immunogenicity, and/or increased serum clearance.

(e) Acidic Variant

The invention also relates to an acidic variant of the anti-IL17A/F antibody either in an isolated form, an enriched form, or in a composition comprising the acidic variant and the main species antibody. The acidic variant can be isolated, characterized, and quantified using various techniques, including, without limitation, imaged capillary isoelectric-focusing (icIEF), ion exchange chromatography (IEC) or pH-Gradient IEC analysis.

In certain embodiments, the amount of acidic variants in a composition is no more than (i.e., equal of less than) about 45%, no more than about 42%, no more than about 40%, no more than about 38%, no more than about 35%, no more than about 32%, no more than about 30%, no more than about 28%, no more than about 25% or no more than about 20%, about 30% to about 42%, about 31% to about 42%, about 32% to about 42%, about 33% to about 42%, about 34% to about 42%, about 35% to about 42%, about 37% to about 42%, about 39% to about 42%, or about 40% to about 42%. In certain embodiments, the amount of an acidic variant in the composition is determined by icIEF. In certain embodiments, the amount of an acidic variant in a composition is no more than about 42% as determined by icIEF. In certain embodiments, the amount of the main peak in the composition is at least about 50%, at least about 54%, at least about 56%, at least about 58%, at least about 59%, at least about 60%, at least about 61%, at least about 63%, at least about 66%, at least about 68%, at least about 70%, at least about 75%, at least about 80%, at least about 85%, at least about 90%, at least about 95% or more. In certain embodiments, the acidic variants in the composition may include one, two, three, four, or five of glycated variant, glycosylation variant, deamidated variant, disulfide reduced variant, sialylated variant, and non-reducible variant. In certain embodiments, the acidic variant is induced by light exposure. In certain embodiments, an anti-IL17A/F antibody composition containing high level of acidic variant exhibits reduced binding and/or reduced neutralizing activity to IL17A, IL17F and/or IL17AF, and/or increased immunogenicity, and/or increased serum clearance.

In general, the amount of the variants present in the composition can be affected by purification. The choice of purification methods can increase or decrease the amount of each variant present in the composition. Commonly used purification methods include, without limitation, protein A affinity column, hydrophobic interaction chromatography, size exclusion column, and ion exchange column chromatography.

(f) Immunoconjugates

In certain embodiments, the composition comprises an immunoconjugates comprising an anti-IL-17A/F antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SLAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A.

### III. Manufacturing and Analytical Methods

According to one embodiment of the invention, a method for evaluating an anti-IL-17A/F antibody composition is provided which comprises one, two, three, or four of: (1) measuring the amount of a glycosylation variant in the composition, and/or (2) measuring the amount of an RR cross-linked variant in the composition, and/or (3) measuring the amount of a HMWS variant and/or LMWS variant in the composition, and/or (4) measuring the amount of an acidic variant in the composition. Optionally, all four analytical assays are performed on a composition comprising the IL-17A/F antibody and variants thereof.

The invention also relates to a method for making a composition comprising: (1) producing a composition comprising the anti-IL-17A/F antibody and one or more variants thereof, and (2) subjecting the composition so-produced to one or more analytical assay(s) to evaluate the amount of the variant(s) therein. The analytical assay(s) can evaluate and quantify the amount of any one or more of: (i) a glycosylation variant and/or (ii) an RR cross-linked variant and/or (iii) a HMWS variant and/or a LMWS variant and/or (iv) an acidic variant. Thus, one, two, three or four of these variants can be analyzed. In certain embodiments, the composition so-produced is protected from light exposure.

In certain embodiments, the analytical assay evaluates, quantifies, or isolates a glycosylation variant, including heterodimer and/or homodimer variants, and/or a HMWS variant, and/or a RR cross-linked variant, and/or an acidic variant. For example, the analytical assay may comprise, without limitation, SE-HPLC, OP-SEC, or icIEF, ion exchange column chromatography, reverse-phase (RP) HPLC, LC/MS, peptide mapping analysis, LC/MS analysis of tryptic mapping, or peptide-N-glycosidase digestion of tryptic mapping followed by LC/MS, capillary electrophoresis-laser induced fluorescence (CE-LIF), 2-amino-benzamide (2-AB) labeling, and 2-aminobenzoic acid (2-AA) labeling.

In addition, the method comprises evaluating the biological activity of an anti-IL-17A/F antibody composition comprising measuring the amount of a glycosylation variant, and/or a HMWS variant, and/or an RR cross-linked variant, and/or an acidic variant in the composition to determine the binding affinity for IL-17A or IL-17F and/or IL-17AF of the composition and/or the inhibitory, neutralizing effects of the IL-17A, IL-17F and/or IL-17AF induced activities of the composition, and confirming the amount of the glycosylation variant, and/or the HMWS variant, and/or the RR cross-linked variant, and/or the acidic variant in the composition is within a respective acceptable range. In certain embodiments, the binding affinity can be determined by, for example, RIA, ELISA, or BIACORE®.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³ M, e.g., from 10⁻⁹M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵1]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd can be measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CMS chips at ˜10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NETS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

The methods optionally further comprise combining the composition with one or more pharmaceutically acceptable excipients to make a pharmaceutical composition. In addition, the pharmaceutical composition can be put into a container which is packaged together with a package insert (e.g. with prescribing information instructing the user thereof to use the pharmaceutical composition to treat cancer) so as to make an article of manufacture.

### IV. Pharmaceutical Compositions

Pharmaceutical compositions comprising the anti-IL-17A/F antibody and one or more variants thereof are prepared for storage by mixing the composition having the desired degree of purity with optional pharmaceutically acceptable excipients (Remington's Pharmaceutical Sciences 16th edition, Osol, A. Ed. (1980)), generally in the form of lyophilized formulations or aqueous solutions. Antibody crystals are also contemplated (see US Pat Appln 2002/0136719). Pharmaceutically acceptable excipients are nontoxic to recipients at the dosages and concentrations employed, and include buffers such as histidine acetate; antioxidants including ascorbic acid and methionine; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbates (e.g. polysorbate 20 or 80), PLURONICS™ or polyethylene glycol (PEG). The pharmaceutical compositions to be used for in vivo administration must be sterile. This is readily accomplished by filtration through sterile filtration membranes.

### V. Therapeutic Applications and Uses

The composition described herein can be administered to an individual in need thereof for treating immune-related or inflammatory diseases or cell proliferation-related diseases such as cancer.

In one aspect, the composition provided herein for use as a medicament is provided. In further aspects, the composition for use in treating immune-related or inflammatory diseases or cell proliferation-related diseases is provided. In certain embodiments, the composition for use in a method of treatment is provided. In certain embodiments, the invention provides the composition described herein for use in a method of treating an individual having an immune-related disease or inflammatory disease or a cell proliferation-related disease comprising administering to the individual an effective amount of the composition. In one such embodiment, the method further comprises administering to the individual an effective amount of at least one additional therapeutic agent, e.g., as described below.

In a further aspect, the invention provides a method for treating an immune-related disease or inflammatory disease or a cell proliferation-related disease. In one embodiment, the method comprises administering to an individual having such immune-related disease or inflammatory disease or a cell-proliferation related disease an effective amount of the composition described herein. In one such embodiment, the method further comprises administering to the individual an effective amount of at least one additional therapeutic agent, as described below.

In a further aspect, the invention provides pharmaceutical formulations comprising any of the compositions provided herein, e.g., for use in any of the above therapeutic methods. In one embodiment, a pharmaceutical formulation comprises any of the compositions provided herein and a pharmaceutically acceptable carrier. In another embodiment, a pharmaceutical formulation comprises any of compositions provided herein and at least one additional therapeutic agent, e.g., as described below.

Compositions of the invention can be used either alone or in combination with other agents in a therapy. For instance, a composition of the invention may be co-administered with at least one additional therapeutic agent. In certain embodiments, an additional therapeutic agent is an antagonist antibody, an agonist antibody, a chemotherapeutic agent or a cytotoxic agent.

Such combination therapies noted above encompass combined administration (where two or more therapeutic agents are included in the same or separate formulations), and separate administration, in which case, administration of the compositions of the invention can occur prior to, simultaneously, and/or following, administration of the additional therapeutic agent or agents. In one embodiment, administration of the compositions of the invention and administration of an additional therapeutic agent occur within about one month, or within about one, two or three weeks, or within about one, two, three, four, five, or six days, of each other. Compositions of the invention can also be used in combination with radiation therapy.

### VI. Article of Manufacture

One embodiment of an article of manufacture herein comprises a container, such as a vial, syringe, or intravenous (IV) bag containing the composition or pharmaceutical composition herein. Optionally, the article of manufacture further comprises a package insert with prescribing information describing how to use the composition according to the previous section herein. In certain embodiments, the article of manufacture is protected from light exposure.

The Examples, which follow, are illustrative of specific embodiments of the invention, and various uses thereof. They are set forth for explanatory purposes only, and are not to be taken as limiting the invention.

### EXAMPLES

### Example 1 Glycosylation Variants of the Anti-IL-17A/F Ab MCAF5352A

The anti-IL17A/F antibody MCAF5352A was produced by Chinese hamster ovary (CHO) cells. The antibody was subjected to size exclusion chromatography (SEC) performed on an Agilent 1100 HPLC system using a Tosoh-Bioscience SEC TSKgel G3000SWx1 (7.8×300 mm, 5 μm) column. Isocratic runtime was 30 minutes at 0.5 mL/min using the mobile phase buffer (0.2 M K₂HPO₄, 0.25 M KCl, pH 6.2) at ambient temperature. 50 μs of antibody diluted in mobile phase buffer was injected for each analysis and monitored at 280 nm. Data was analyzed using Chromeleon Software package (Dionex).

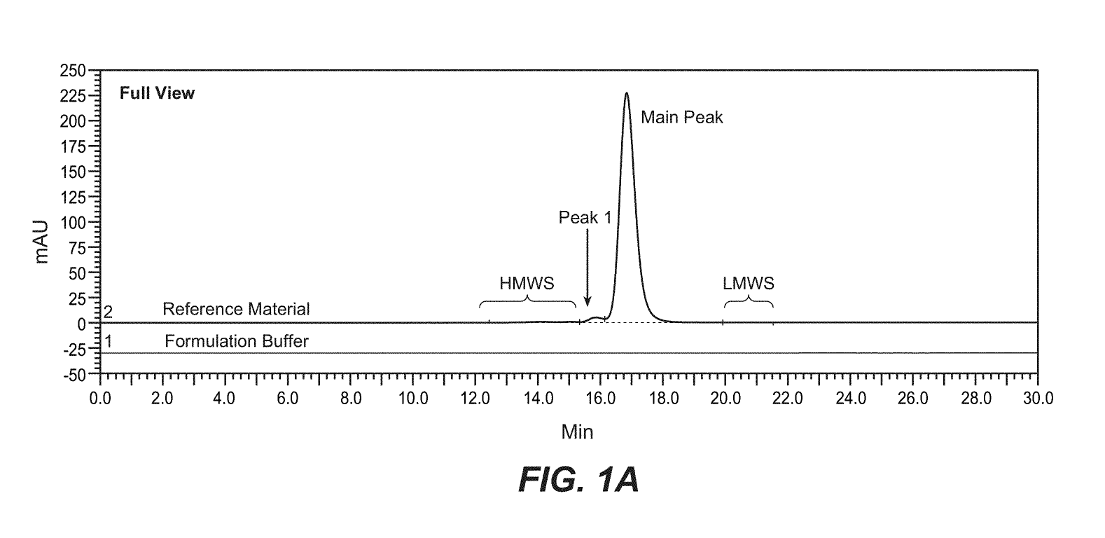

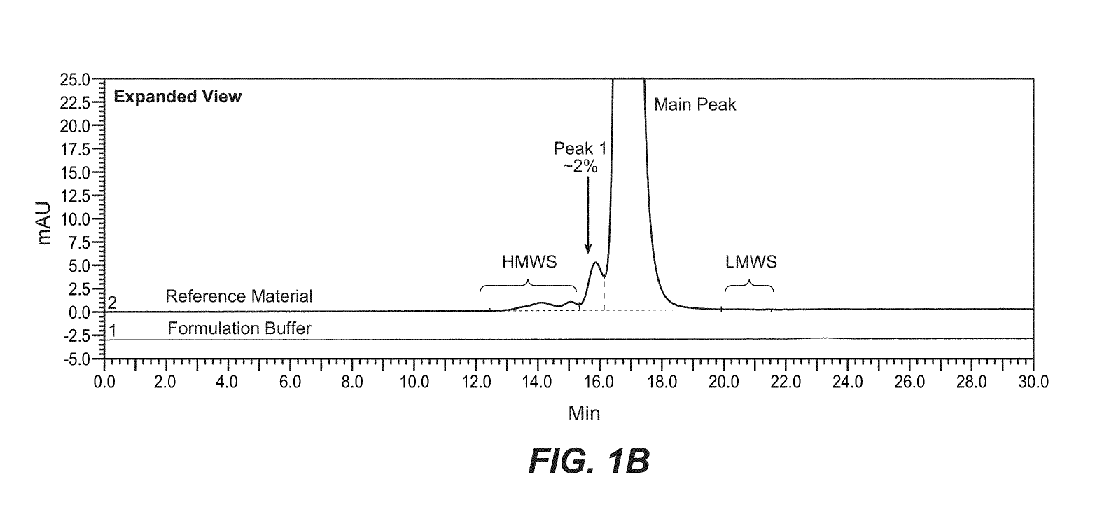

As shown in FIG. 1A and FIG. 1B, an additional peak (Peak 1) was apparent in the SEC analysis, with a molecular weight slightly larger than the main Ab peak. The LC-MS (liquid chromatography-mass spectrometry) data confirmed that Peak 1 is a heterogeneous mixture of species with mass additions ranging from approximately 2400-3000 Da (data not shown). The mass additions have been localized to the Fab heavy chain region (data not shown). The SEC analysis of samples shows that the composition comprises about 2% of Peak 1. Peak 1 was resistant to DTT treatment (data not shown).

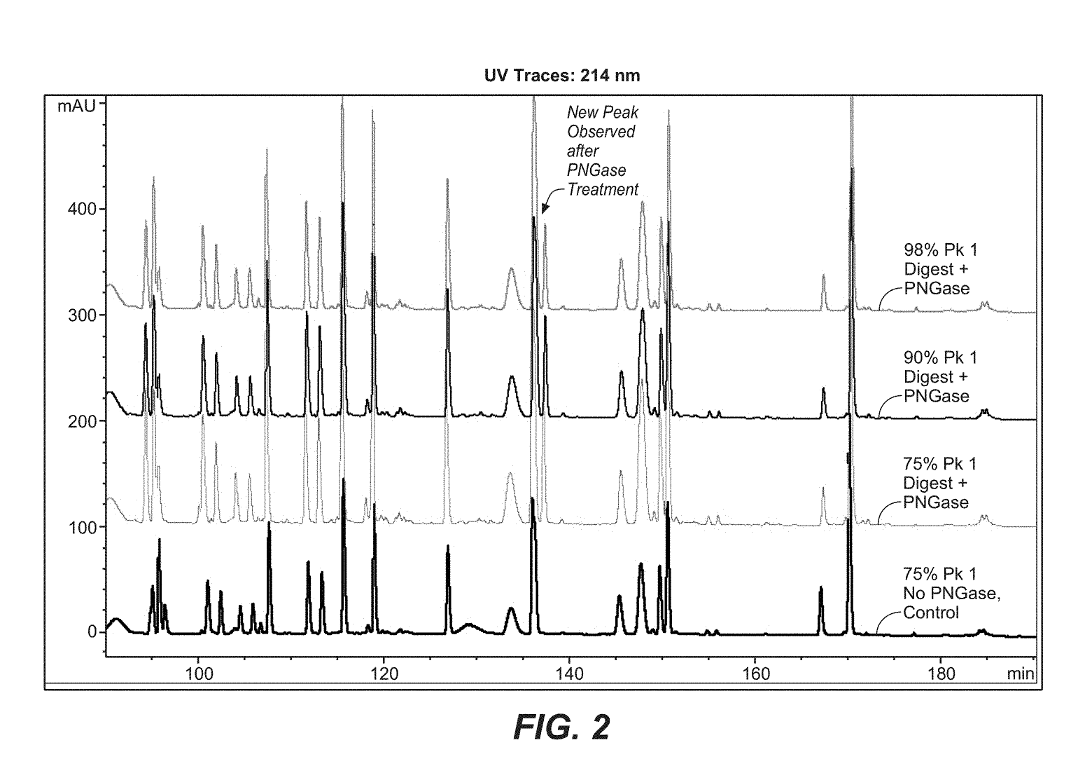

Peptide mapping LC-MS data suggested that N-linked glycosylation accounted for the presence of Peak 1 (data not shown). Enriched Peak 1 samples were treated with trypsin followed by PNGase (Peptide-N-Glycosidase, New England Biolabs) at 37° C. overnight. As shown in FIG. 2, a new peak appeared after PNGase digestion, indicating N-linked glycosylation in peptide HC44-65.

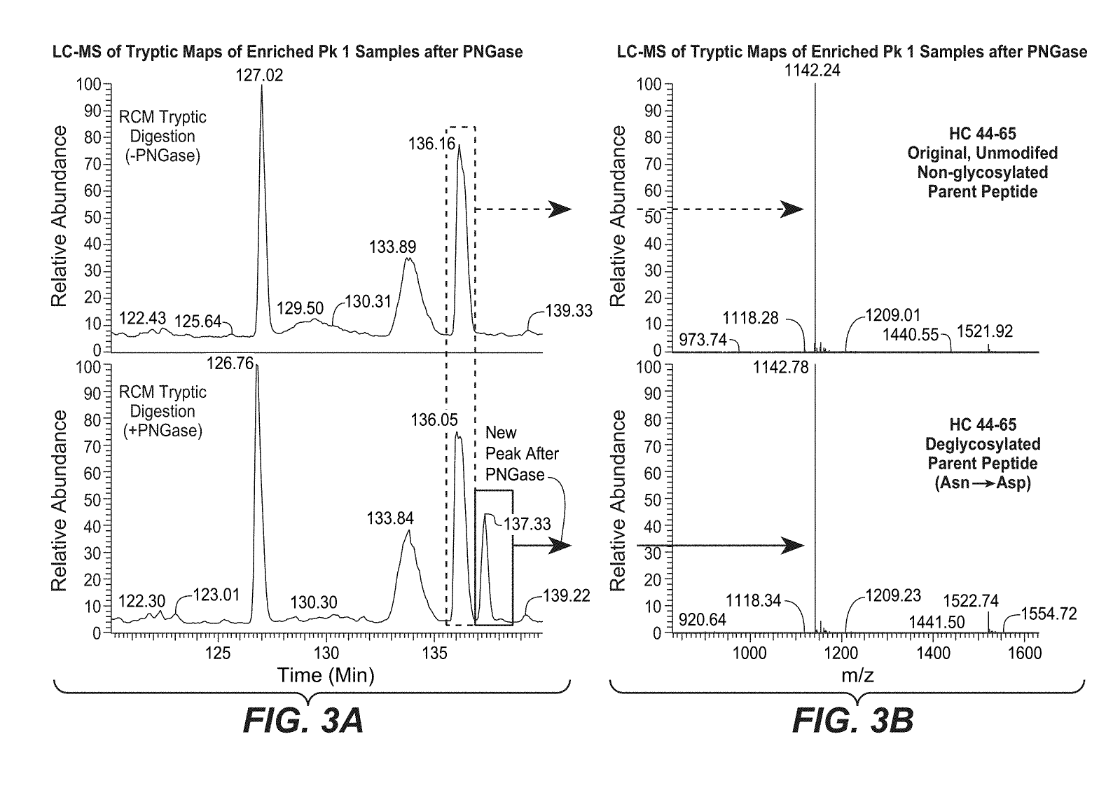

Next, the glycosylation site on the Fab was determined. The tryptic peptide HC 44-65 contains the sequence GLEWVSGINWSSGGIGYADSVK (SEQ ID NO:11, HC CDR2 sequence underlined), of which NWS represents a consensus sequence for N-linked glycosylation. LC-MS analysis of tryptic map of enriched Peak 1 showed that the mass of the additional peak after PNGase treatment slightly increased, consistent with the conversion of Asn to Asp as a result of PNGase digestion. See FIG. 3A-B. The presence of Asp52 in the deglycosylated HC44-65 was confirmed by N-terminal sequencing of the collected peptide. Approximately 30 different glycopeptide masses were observed in the mass spectra, and accurate mass structural assignments showed that many of the glycans are likely sialylated, which corroborates with the acidic nature of Peak 1 (data not shown).

The composition of anti-IL-17A/F antibody that comprises about 90% glycosylation variant in the HC CDR2 showed reduced potency. IL-17A/F binding by the Peak 1 or glycosylation variant-enriched antibody samples was compared with sample with composition with 2% glycosylation variant. Varying concentrations of the antibody standard, control, and samples were added to 96-well plates coated with either IL-17 AA or IL-17 FF or IL-17AF. Bound IL-17A/F antibody was detected with anti-human-HRP and a TMB substrate solution. The results, expressed in OD, were plotted against the IL-17A/F antibody concentrations, and a 4-parameter curve-fitting program is used to estimate the activity of the anti-IL17A/F antibody sample(s) relative to the Reference Material. Results are reported as relative potency, assigning Reference Material that comprises 2% of the glycosylation variant as 100%.

As shown in Table 2 below, the presence of glycosylation in HC CDR2 greatly reduced binding to IL-17AA, IL-17FF and IL-17AF as measured by ELISA.

TABLE 2

| ELISA | Specific Activity (n = 2) |
|---|---|
| Binding to AA | 57% |
| Binding to AF | 69% |
| Binding to FF | 67% |

The results show that sample containing anti-IL-17A/F antibody with more than 2% glycosylation variants exhibit much reduced binding activity as compared to sample with 2% glycosylation variants.

### Example 2 Photo-Induced Discoloration of the Anti-IL17A/F Ab MCAF5352A is Linked to HMWS Formation

The anti-IL-17A/F antibody MCAF5352A exhibits atypical photo-sensitivity that can lead to discoloration (yellowing), reduction-resistant (RR) cross linking, and HMWS formation from exposure to light, such as ambient laboratory lighting. To further understand the photo-sensitivity properties, the anti-IL-17A/F antibody was subjected to light exposure under ICH guideline conditions (Sun Test) and evaluated by charge variant analysis, size exclusion analysis, peptide mapping, and mass spectrometry analysis.

Light Stressed Sample Preparation

Antibody samples were prepared using an Atlas Suntest CPS+ light box using the following ICH guideline conditions: Irradiance level=250 watts/sq meter, Total UV dose=538 watt-hours/sq meter, Total Visible dose=1,320,000 lux-hours/sq meter. Exposure times were as indicate.

Charge Variant Analysis Using Imaged Capillary Isoelectric-Focusing (icIEF) Analysis

Charge variant analysis was performed using the iCE280 Analyzer with a FC-coated fluorocarbon capillary, 100 μm id×5-cm long (PN101701, Protein Simple, San Jose, Calif.). The ampholyte solution was preparation as follows: 700 μL of 1% Methyl Cellulose Solution (Protein Simple, Santa Clara, Calif.), 237 μL purified H₂O, 1 mL 5 M urea, 44 μL Pharmalyte 8-10.5 (GE Healthcare), 19 μL Pharmalyte 5-8 (GE Healthcare, Waukesha, Wis.), 8 μL pI Marker 5.5 (Beckman Coulter, Brea, Calif.), 8 μL pI Marker 9.77 (Convergent Bioscience, Toronto, ON). 160 of ampholyte solution was mixed with 40 μL of 1 mg/mL antibody post carboxypeptidase (CpB) treatment (1:100 CpB to antibody, 37° C. for 20 minutes). Focusing condition were 1500 V for 1 min, followed by 3000 V for 10 minutes, prior to 280 nm absorbance detection.

Size Exclusion Chromatography (SEC)

Size exclusion chromatography (SEC, also referred to as SE-LC or SC-HPLC) was performed on an Agilent 1100 HPLC system using a Tosoh-Bioscience SEC TSKgel G3000SWx1 (7.8×300 mm, 5 μm) column. Isocratic runtime was 30 minutes at 0.5 mL/min using the mobile phase buffer (0.2 M K2HPO4, 0.25 M KCl, pH 6.2) at ambient temperature. 50 μg of the antibody diluted in mobile phase buffer was injected for each analysis and monitored at 280 nm. Data was analyzed using Chromeleon Software package (Dionex, Sunnyvale, Calif.).

Organic Phase Size Exclusion Chromatography (OP-SEC)

Organic Phase SEC was performed on an Agilent 1200 HPLC system using a two Tosoh-Bioscience size exclusion chromatography TSKgel SuperSW3000 (2×300 mm, 4 μm) columns linked in series. Isocratic runtime was 45 minutes at 0.25 mL/min using an organic mobile phase buffer (60% ACN (acetonitrile) in H₂O, 0.1% TFA (trifluoroacetic acid)) at 70° C. All samples were prepared at 1 mg/mL concentrations in 1 mL of 20 mM Tris (pH 7.5) buffer. Reduction of the mAb was performed by incubating samples in 10 mM DTT at 37° C. for 15 minutes, followed by the addition of 20 μL of 0.1% TFA to prevent disulfide-bond reformation. 25 μg of mAb was injected for each analysis and monitored at 280 nm. Data was analyzed using Chromeleon Software package (Dionex).

A QTOF Premier mass spectrometer (Waters, Milford, Mass.) was operated in positive electrospray ionization mode and coupled on-line to the HPLC system for the OP-SEC-MS analysis. Instrument control and data analysis were performed using a Waters MassLynx software package (version 4.1) (Milford, Mass.). Deconvolution of multiply charged ions was performed with the MaxEnt 1 software provided with MassLynx.

Capillary Electrophoresis-Sodium Dodecyl Sulfate (CE-SDS)

Capillary Electrophoresis-Sodium Dodecyl Sulfate (CE-SDS) was performed as follows. Samples were derivatized with 5 carboxytetramethylrhodamine succinimidyl ester, a fluorescent dye. After removing the free dye through gel filtration (using NAP-5 columns), nonreduced samples were prepared by adding SDS/40 mM iodoacetamide and heating at 70° C. for 5 min. For the analysis of the reduced samples, the derivatized samples were mixed with SDS to a final concentration of 1% (v/v) and 10 μL of a solution containing 1 M dithiothreitol, and heated at 70° C. for 20 min. The prepared samples were analyzed on a Beckman Coulter ProteomeLab PA800 system using a 50 μm internal diameter 31.2 cm fused silica capillary maintained at 20° C. throughout the analysis. Samples were introduced into the capillary by electrokinetic injection at 10 kV for 40 s. The separation was conducted at a constant voltage of −15 kV using CE-SDS running buffer as the sieving medium. An argon ion laser operating at 488 nm was used for fluorescence excitation with the resulting emission signal monitored at 560 nm.

RCM (Reduction c-CarboxyMethylation) Tryptic Peptide Mapping and RP-HPLC-MS Analysis

Full-length antibody samples were diluted into denaturing buffer (6 M Guanidine, 360 mM Tris, 2 mM EDTA, pH 8.6) and reduced by incubation at 45° C. for 10 minutes in the presence of 10 mM DTT. S-carboxymethylation was performed to cap the cysteines after reduction by incubating samples at 45° C. for 10 minutes in the presence of 20 mM sodium iodoacetate, then quench at room temperature with 40 mM DTT. Samples were then desalted by loading onto NAP-5 columns (GE Healthcare) and eluted with 800 uL of trypsin digest buffer (20 mM Tris pH 8.0). Samples were digested at 37° C. with 3% trypsin (w/w, Roche Life Science) for 3.5 hours. TFA was added to a final concentration of 0.3% to stop the digest. Tryptic peptides were separated using an Agilent 1200 HPLC with PHENOMENEX JUPITER™ C-18 column (2×250 mm, 5 μm, 300 Å). Peptide elution was performed using a gradient from 100% solvent A (H₂O, 0.1% TFA) to 45% solvent B (ACN, 0.1% TFA) over 215 minutes at a flow rate of 0.25 mL/min. Mass spectrometric analysis of chromatographic peaks observed at 214 nm was performed with a ORBITRAP ELITE™ mass spectrometer (Thermo Fisher Scientific) operating in the positive ion mode. Data analysis was performed with Thermo Excalibur software.

Intact and Reduced Mass Analysis

The purpose of this assay was to confirm the molecular weight of the intact antibody, and the molecular weights of the heavy chain and light chain of the reduced antibody. Samples were analyzed using the Agilent ESI-TOF (ChipTOF) using a Protein Chip (II) 43 mm×75 Zorbax 300SB-C8, 5 μm column. Intact samples were prepared at 0.1 mg/mL in 5% Acetonitrile/0.1% Formic Acid, and analyze using the Aglient ESI-TOF. Reduced samples were treated with 20 mg/mL TCEP at 60° C. for 10 min, and then prepared at 0.05 mg/mL in 5% Acetonitrile/0.1% Formic Acid, and analyze using the Aglient ESI-TOF. Samples were injected at 0.05 μL for analysis. Intact and Reduced masses were deconvoluted using Mass Hunter Software (Agilent Software Suit).

Cross-Linked Peptides Identification Using O¹⁸-Labeling

Samples were prepared and analyzed as described above with the following exception. After the NAP-5 desalting procedure the samples were split in half and speed-vac to dryness. The samples were then reconstituted in either LC-MS grade H₂O′⁶ or H₂O¹⁸ (99.7% atom purity) and trypsinized as described above with lyophilized trypsin reconstituted in the appropriate H₂O. The RP-HPLC analysis was performed as above. All parent ions were fragmented using high collision-induced dissociation (HCD) and the associated transitions were detected using the Fourier Transfer (FT) analyzer for high mass accuracy analysis.

Scrambled Disulfide Cross-Linking Using Native Tryptic Mapping

Samples were diluted into denaturing buffer (7 M Guanidine, 0.1 mM Sodium Acetate, 10 mM N-Ethyl Maleimide (NEM), pH 5.5) and incubated at 37° C. for 2 hours. Samples were then desalted by loading onto NAP-5 columns (GE Healthcare) and eluted with 700 uL of trypsin digest buffer (0.1 M Tris, 1 mM Calcium Chloride, pH 7.5). Samples were digested at 37° C. with 10% trypsin (w/w, Roche recombinant) overnight in the presence of 10% acetonitrile. Digested samples were split in half (400 μL) for reduction in the presence of 15 mM Tris(2-carboxyethyl)phosphine (TCEP) at 37° C. for 30 min. The addition of 25% TFA was added to both the non-reduced and reduced samples.

Results

Light Exposure Leads to Photo-Induced Discoloring of Anti-IL-17A/F Antibody and Reduction-Resistant HMWS Formation

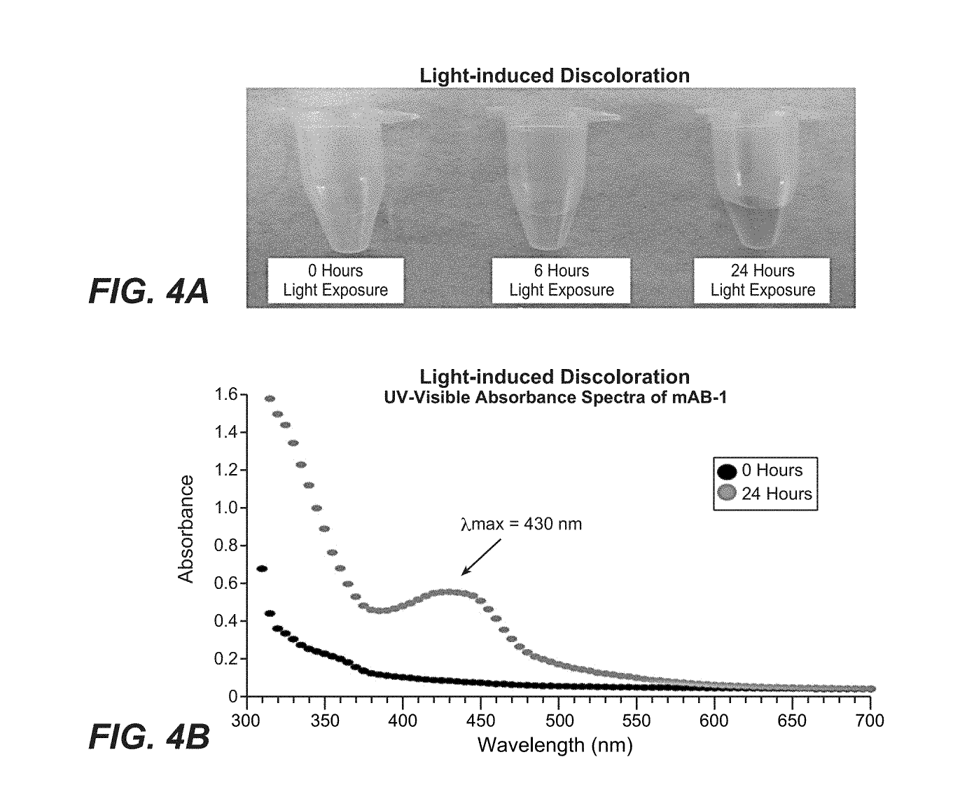

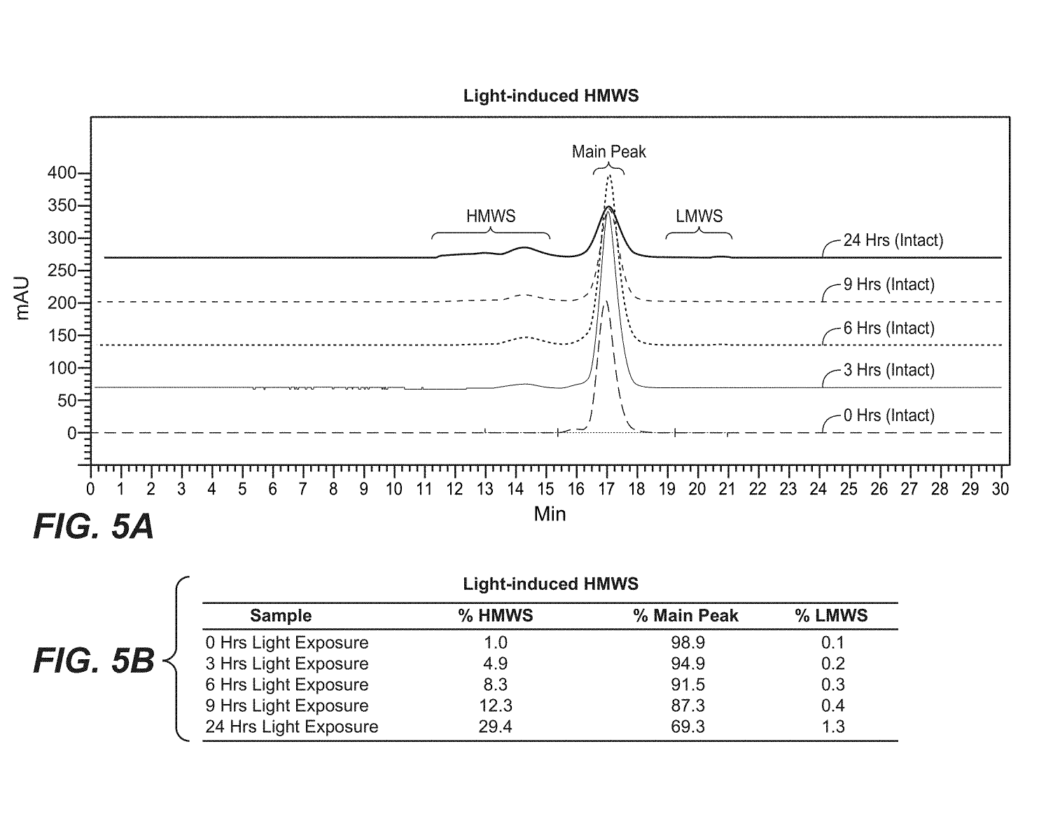

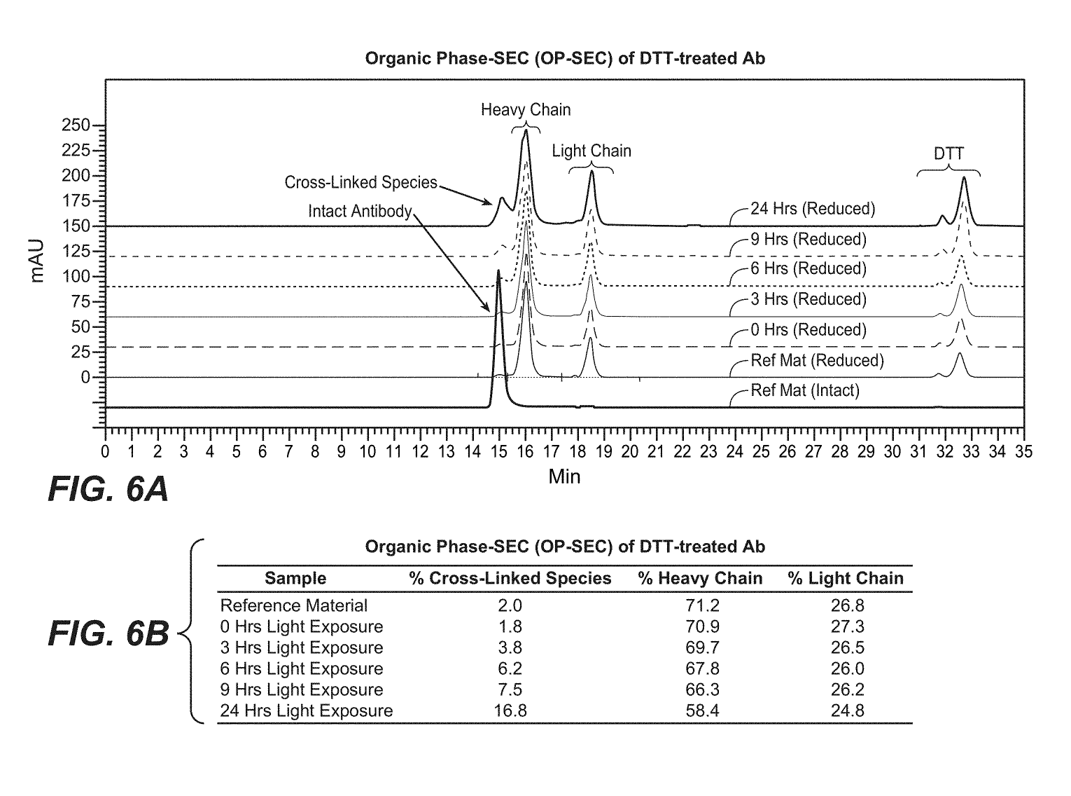

As shown in FIG. 4, anti-IL-17A/F antibody MCAF5352A exposed to light exhibited a noticeable photo-induced yellowing with an Amax˜430 nm. The photosensitivity was observed to both ambient laboratory light and ICH Guidelines light conditions. The absorbance is considerably redshifted compared to the expected absorbance due to tryptophan oxidation (absorbance between 315 nm-370 nm). Samples exposed to light at varying time points were first analyzed using a standard SEC method to determine the extent of HMWS formation in the intact protein. The SEC data demonstrated there was a linear increase in photo-induced aggregations, reaching nearly 30% HMWS at the 24 hour time point (FIG. 5). Oxidation of peptide side chains can affect local environment of a protein, leading to the exposure of hydrophobic sections and non-specific aggregation. To investigate the nature of the observed aggregation, a novel organic phase SEC (OP-SEC) method was developed to analyze the reduced components of the light exposed samples. As seen in FIG. 6, light-exposed anti-IL17A/F sample contained an apparent non-reducible species that eluted between the intact protein and the heavy chain. This species was observed to increase linearly with light exposure (FIG. 6B) in a similar manner as the HMWS seen in the SEC (FIG. 5B). At 24 hours light exposure, this reduction-resistant or non-reducible HMWS (RR-HMWS or NR-HMWS) reaches ˜16% of the total species observed. The presence of RR-HMWS was also confirmed by CE-SDS (data not shown).

Light Exposure Increases Acidic Charge Variants of Anti-IL17A/F Antibody MCAF5352A

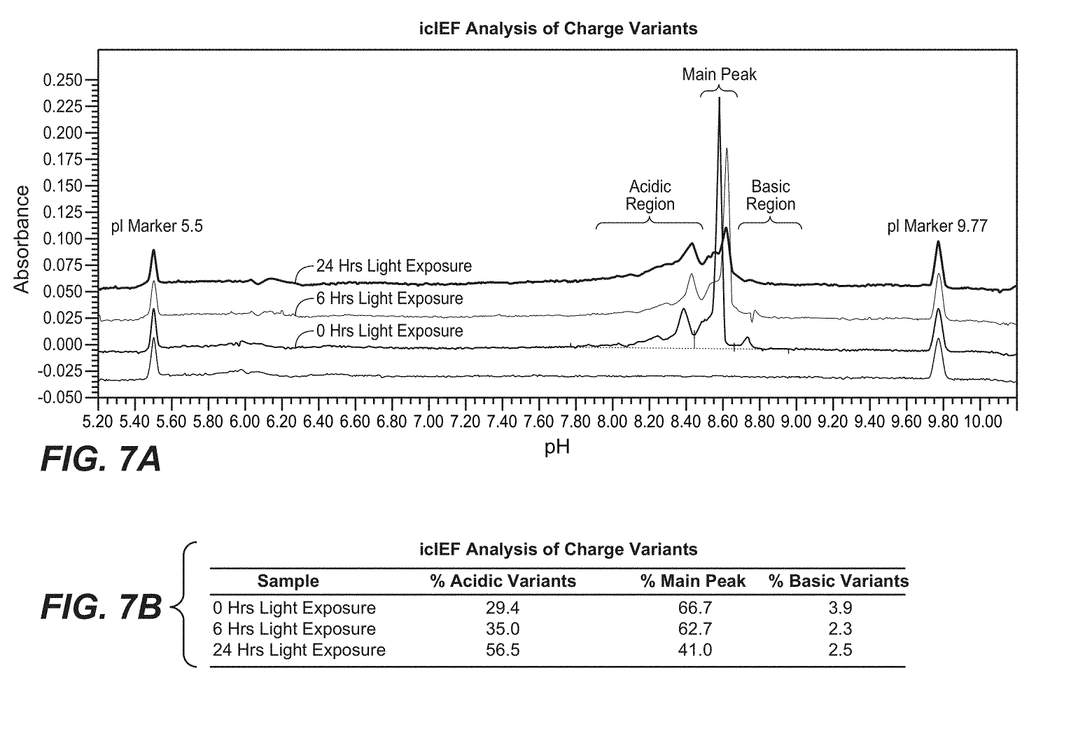

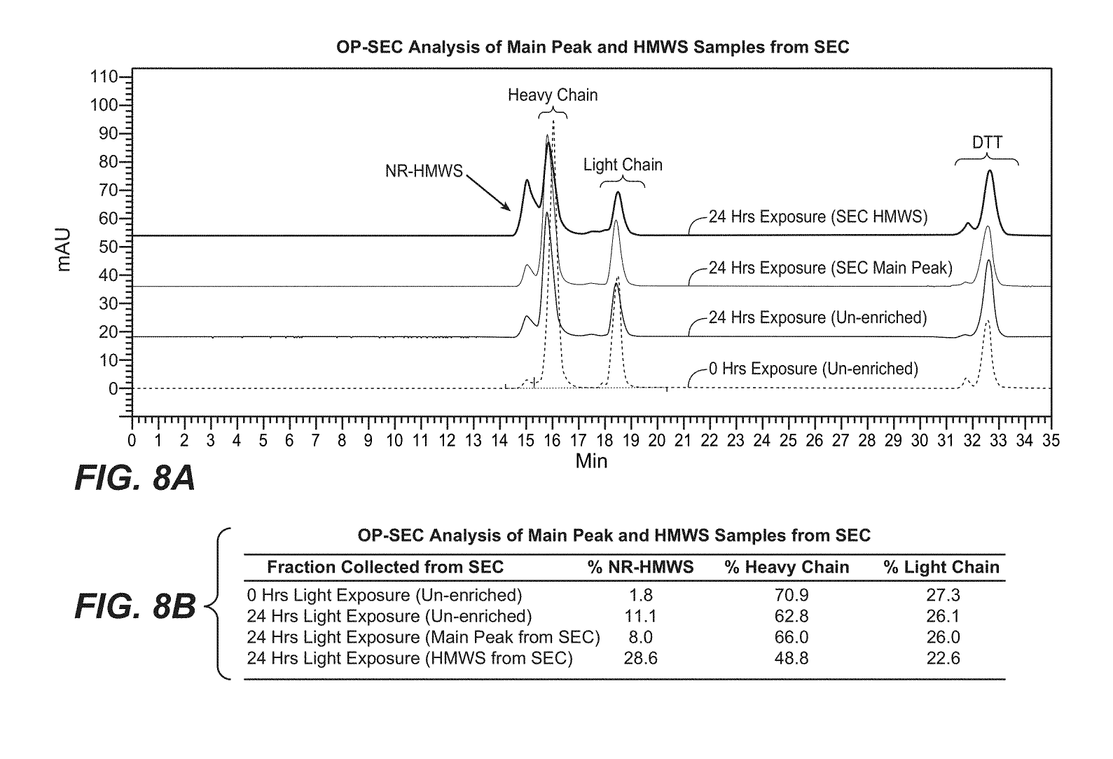

To understand the global effects of light exposure on the IL-17A/F antibody, we performed icIEF analysis to monitor for changes in charge variants. The icIEF analysis demonstrates a significant increase in acidic variants upon light exposure, with little effect on the basic variants (FIG. 7). The icIEF data confirms that there is no increase in basic charge variants. Without being limited to one or more mechanisms, the acidic variants are likely linked to Met and/or Trp oxidation.

The Light-Induced HMWS Contains Both Inter-Molecular and Intra-Molecular Cross-Links

To determine if the NR-HMWS observed from the OP-SEC method were from inter-molecular cross-linking, we collected the intact HMWS aggregates and the main peak from the SEC assay, and analyzed these fractions using the OP-SEC method. The HMWS fraction from the SEC showed a nearly 3-fold enrichment in RR-HMWS, while the main peak fraction from the SEC had a decrease in the overall RR-HMWS (FIG. 8). This data indicates that both inter- and intra-molecular cross-linking were occurring after light exposure; however, there is significantly more cross-linked species in the SEC-collected aggregates than the SEC-collected main peak, suggesting primarily inter-molecular cross-linking.

Methionine and Tryptophan Oxidation are Detected by Tryptic Peptide Mapping after 24-Hour Light Stress

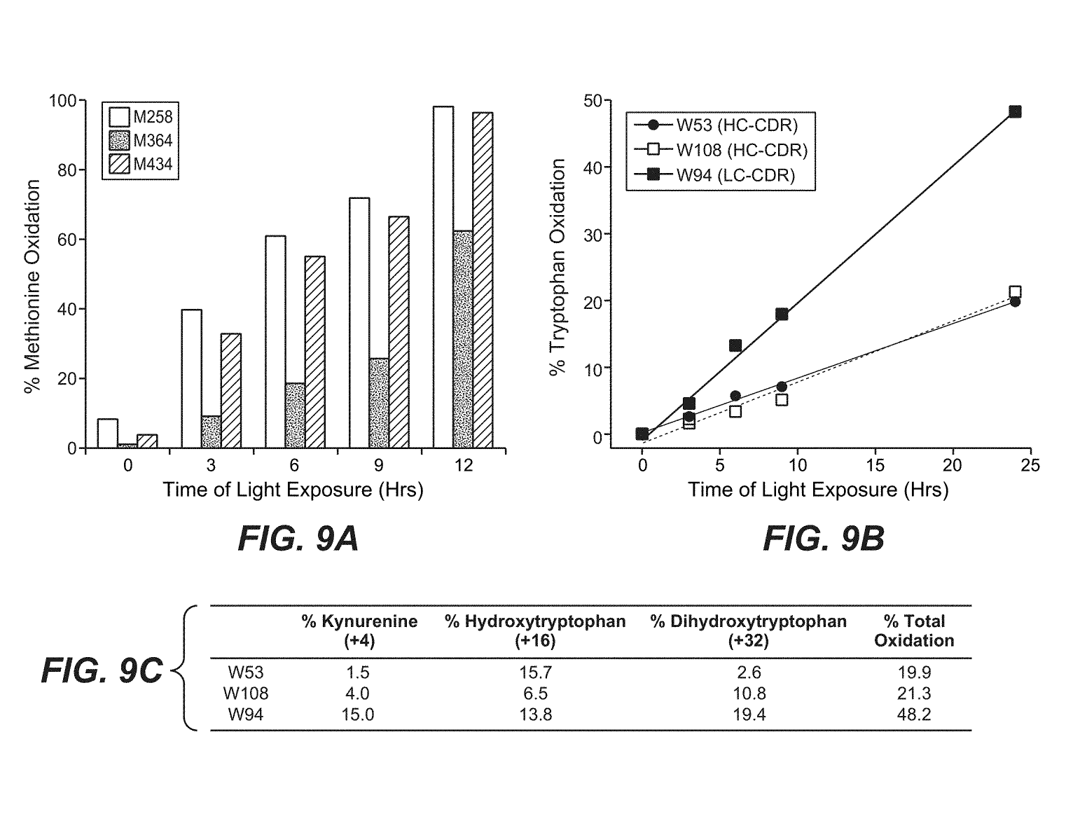

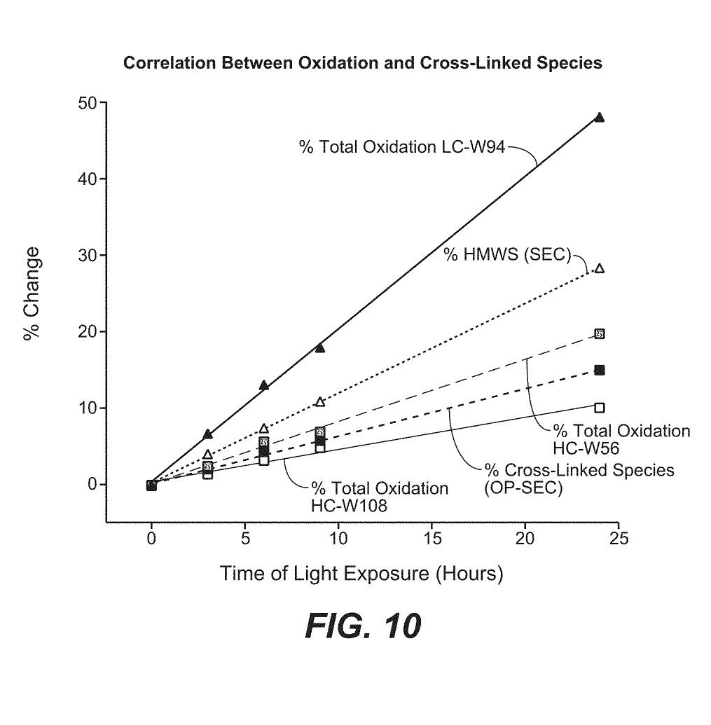

Tryptic digests were analyzed using LC-MS/MS, and extracted ion chromatograms (XIC) were used to quantify the amount of Met and Trp oxidation in oxidized peptides relative to native peptides. The most susceptible residues to photo-induced oxidation were the three Met residues found in the FC region (M258, M364, M434 according to SEQ ID NO:9) (FIG. 9A). Three Trp residues, two in the CDR of the HC (W53 and W108 according to SEQ ID NO:7 or SEQ ID NO:9) and one in the CDR of the LC (W94 according to SEQ ID NO:8 or SEQ ID NO:10) exhibited extensive oxidation (FIG. 9B). All three Trp residues exhibited a linear increasing in the overall oxidation, but each displayed varying amounts of the individual oxidation species (FIG. 9C). Although the overall extent of oxidation for W53 and W108 was nearly identical, the rate of dihydroxytryptophan conversion was 4-fold higher for W108 (0.45% ox/hr vs. 0.11% ox/hr for W108 and W53, respectively). W94 is the most susceptible Trp residue with nearly 2.5-fold more overall oxidation than W53 and W108. In addition, W94 exhibited a significant amount of kynurenine species compared to the other two Trp residues. As shown in FIG. 10, the three tryptophans (LC W94, HC W56 ad HC W108) display significant susceptibility to photo-induced oxidation. Light-induced increase in these variants also corresponded to the increase in overall HMWS formation and RR cross linked species, and the qualitative increase in coloring.

Light-Induced HMWS Contains Predominantly Heavy Chain-Heavy Chain Cross-Linked Variant and to a Less Extent Heavy Chain-Light Chain Cross Linked Variant

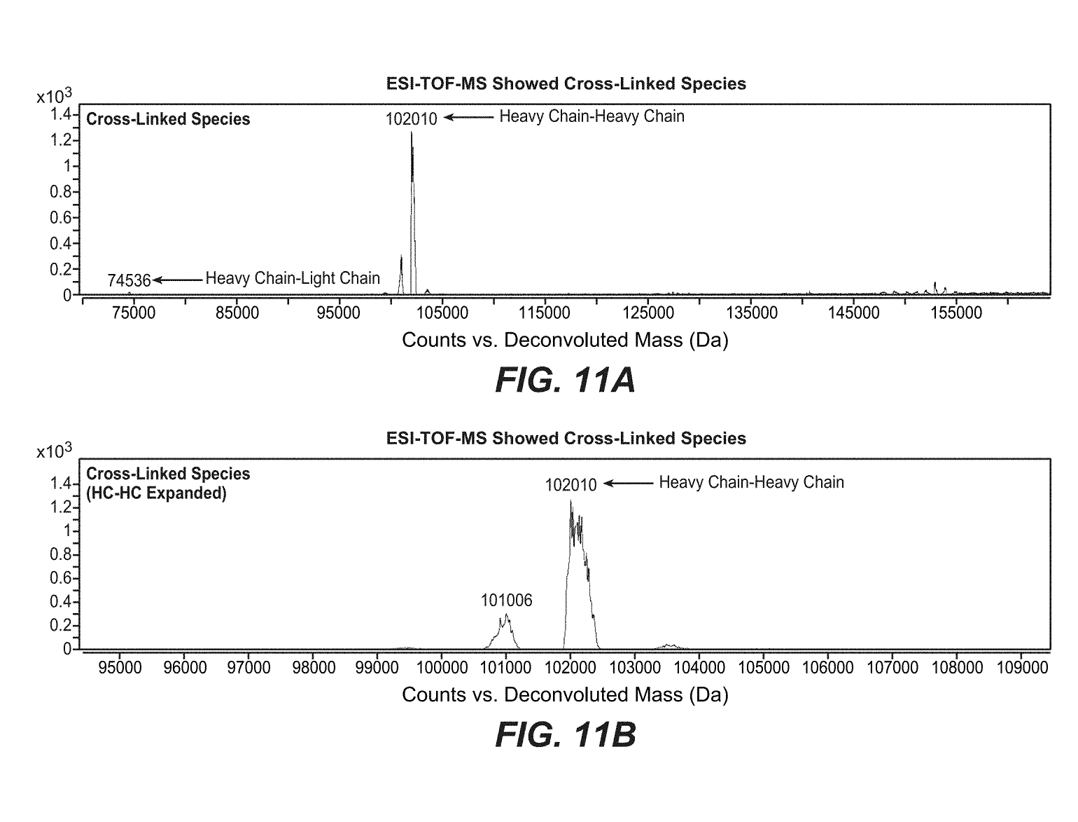

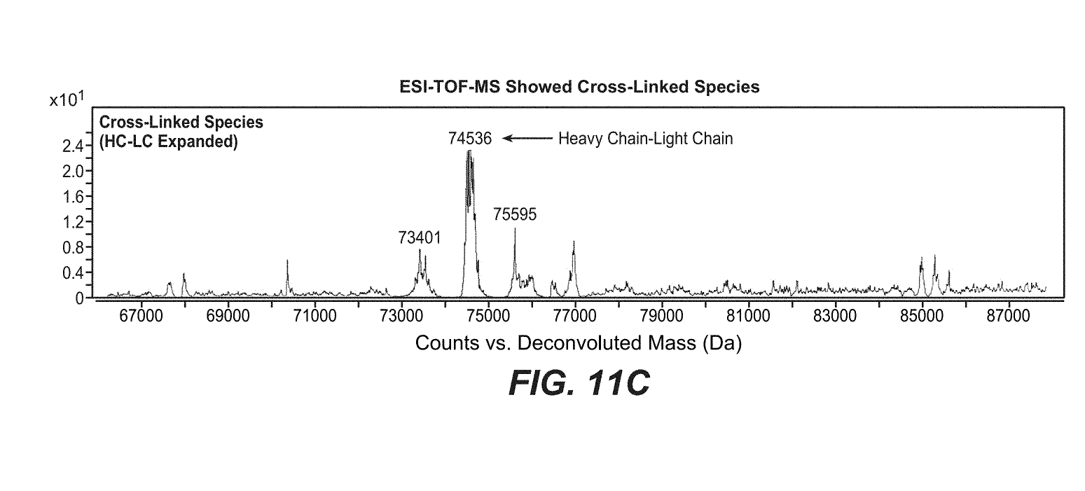

Fractions of OP-SEC light stressed samples were collected for more precise analysis using ESI-TOF-MS. The deconvoluted ESI-TOF-MS data show that there was significant oxidation in both the HC and LC of anti-IL-17A/F antibody MCAF5352A after 24 hours of light exposure, and that the RR-HMWS fraction contained primarily a species with a mass of ˜102 kDa, and to a lesser extent, a species with a mass of ˜74.5 kDa (FIG. 11B-C). These results were consistent with the OP-SEC-MS data and suggestive of both HC-HC and HC-LC covalent cross-linking.

### Example 3 Photo-Induced Coloring and HMWS Formation is a Reactive Oxygen Species (ROS)-Driven Process

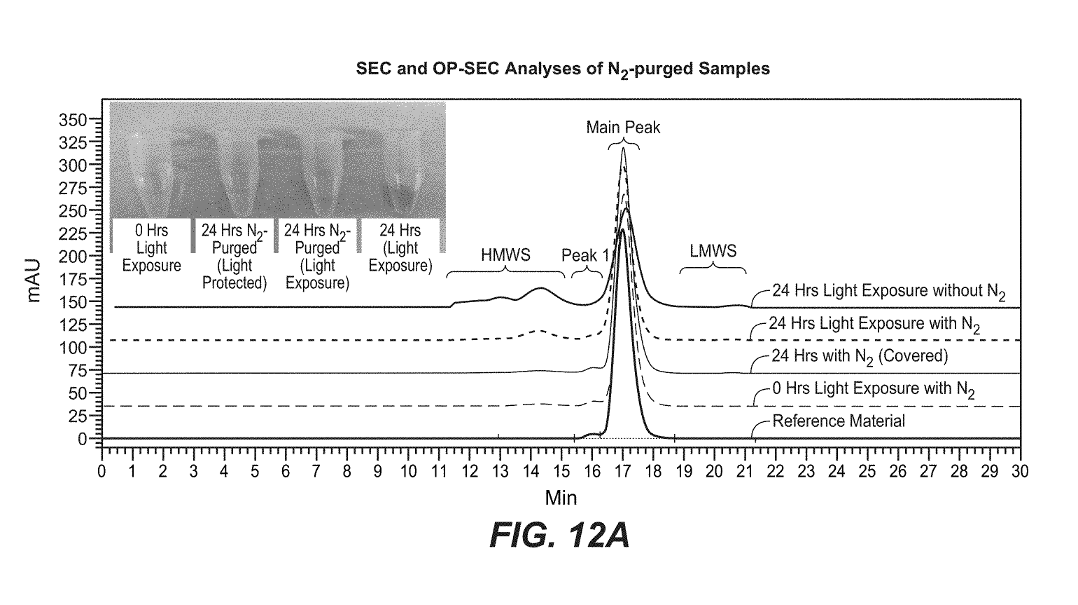

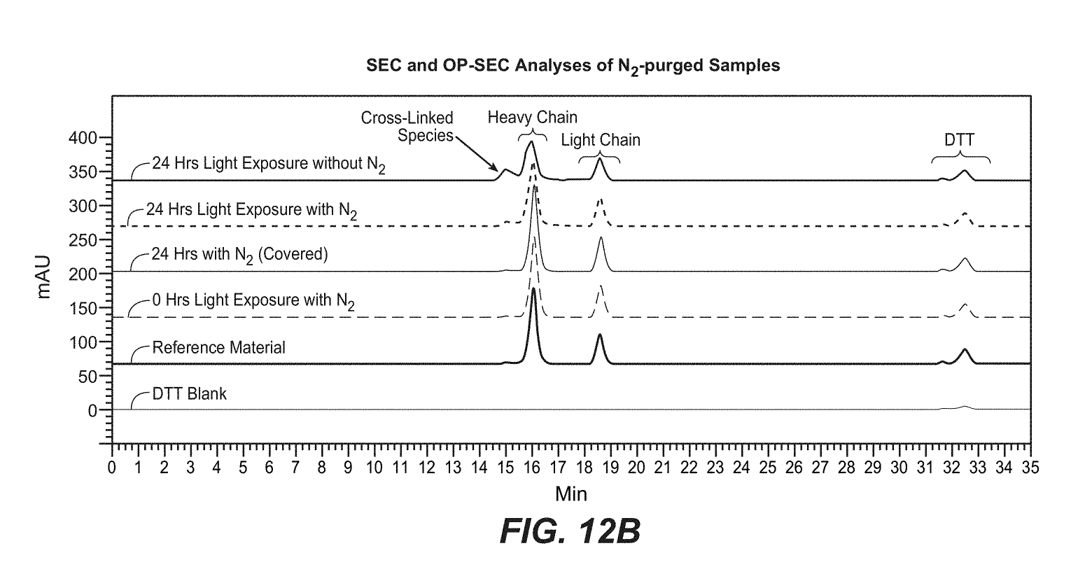

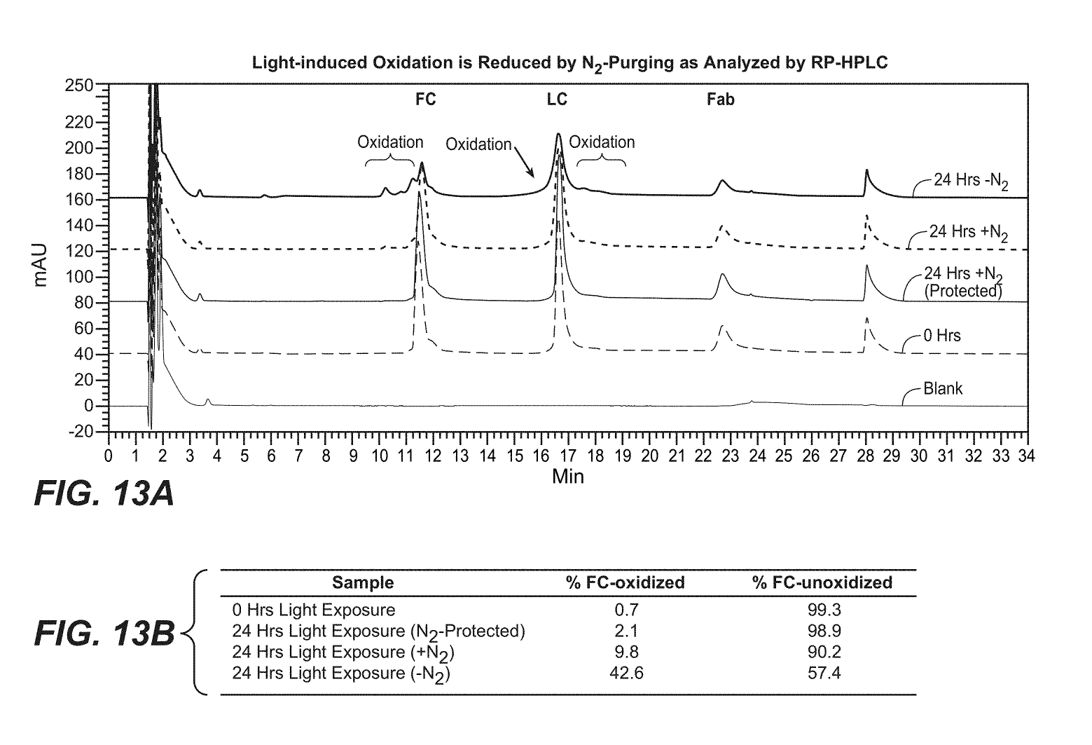

To first investigate if the photo-sensitivity of anti-IL-17A/F antibody was due to oxidation, the antibody sample was purged with N₂ gas prior to 24 hours light exposure. A visible decrease in the extent of discoloration was observed with the N₂-purged sample, coupled with a reduction in both HMWS and cross-linked species (FIG. 12). This correlated with a reduction in global oxidation as analyzed by RP-HPLC oxidation assay (see below and FIG. 13).

The detection and quantitation of global oxidation was performed using a reverse phase (RP)-HPLC assay. Samples were prepared at 1 mg/mL concentrations in 50 mM Tris pH 8.0, and digested with FabRICATOR® (IdeS) (Genovis, Cambridge, Mass.) (50 unit per 100 μg of antibody) for 4 hours at 37° C. Digested samples were then reduced with 20 mM DTT (8 M Guanidine, 50 mM Tris pH 8.0) for 30 min at 37° C. TCEP (Tris(2-carboxyethyl)phosphine) was then added to a final concentration of 25 mM prior to analysis. Reduced digest were separated using an Agilent 1200 HPLC with BioBasic Phenyl™ Column (2.1×150 mm, 5 μm, 300 Å) (Thermo Fisher Scientific, Waltham, Mass.). Peptide elution was performed using a gradient from 68% solvent A (H₂O, 0.1% TFA) to 55% solvent B (ACN, 0.1% TFA) over 19 minutes at a flow rate of 0.3 mL/min.

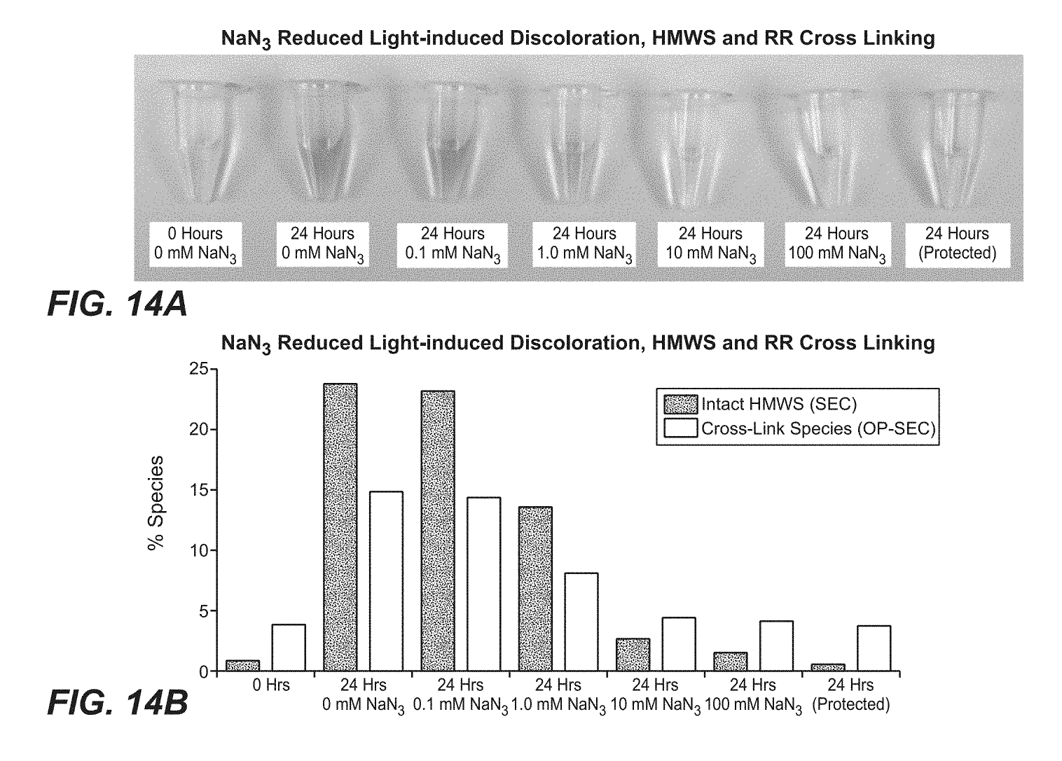

The sample was exposed to light in the presences of concentrations of NaN₃ ranging from 0.1-100 mM to investigate the involvement of ROS. It was observed that NaN₃ provided a protective effect on the photo-oxidation of the antibody; however, there was also a direct correlation between the concentration of NaN₃ and the extent of discoloration and RR HMWS formation/RR Cross-link formation (FIG. 14). In conclusion, the antibody displays a unique photosensitivity to both ambient laboratory light and ICH Guidelines' Light conditions which results in a strong absorbance in the visible region with a λmax ˜430 nm. This absorbance is considerably redshifted compared to the expected absorbance due to tryptophan oxidation (absorbance between 315 nm-370 nm). The addition of NaN3 provided a protective effect by reducing the 430 nm absorbance in a dose-dependent manner, strongly indicating that this unique absorbance is a byproduct of photo-induced singlet oxygen-derived reactive oxygen species, the formation of cross-linked species was a result from photo-induced singlet oxygen, and discoloration directly related to the cross-linked species.

### Example 4 Identification of Cross-Linked Peptides Using O¹⁸-Labeling

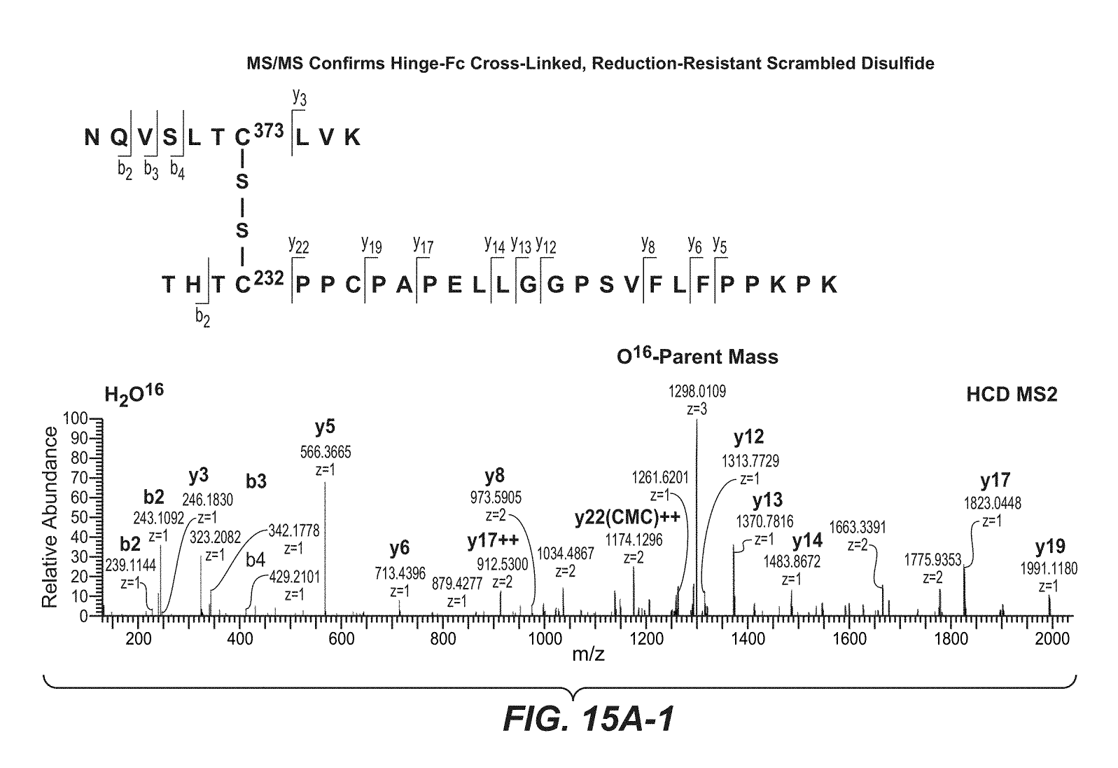

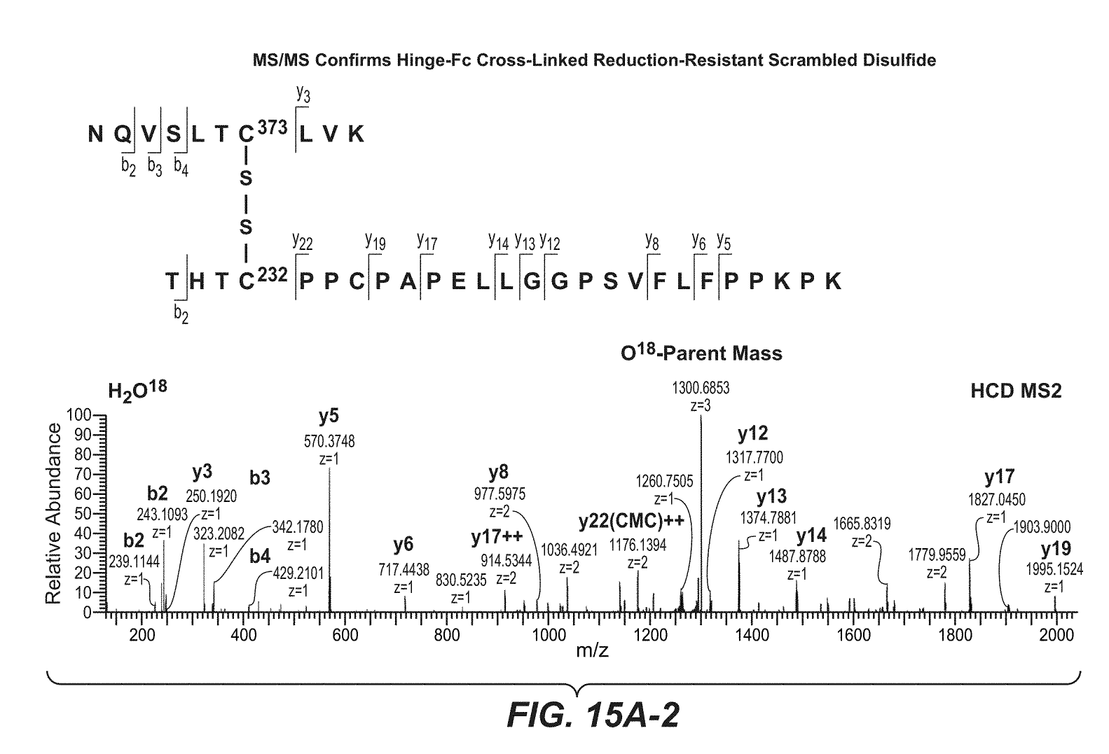

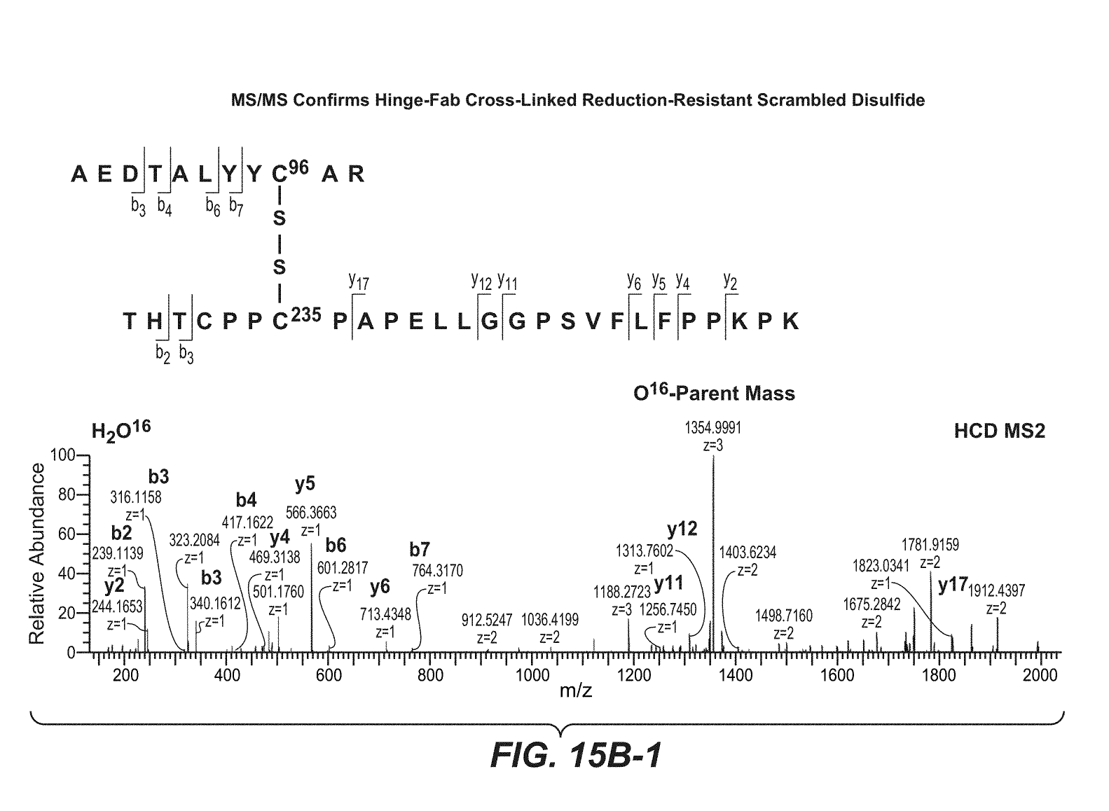

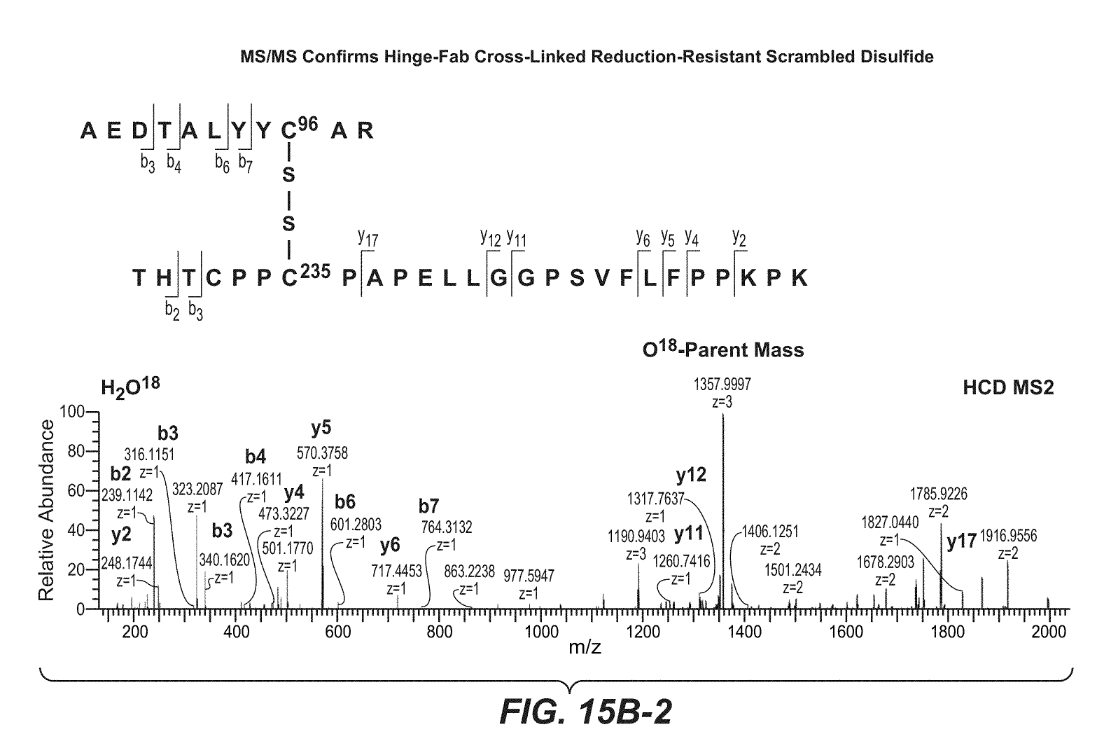

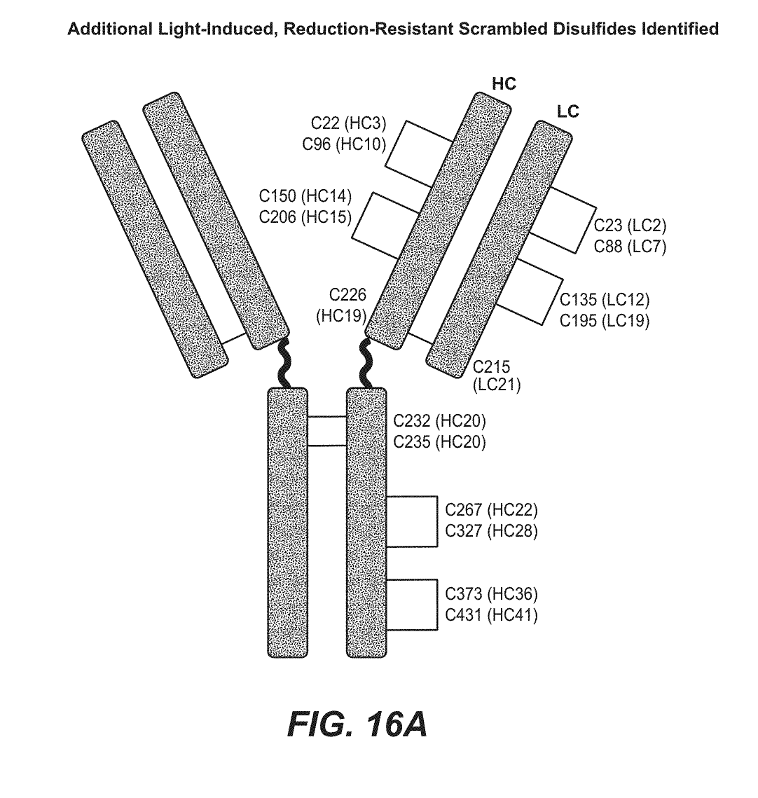

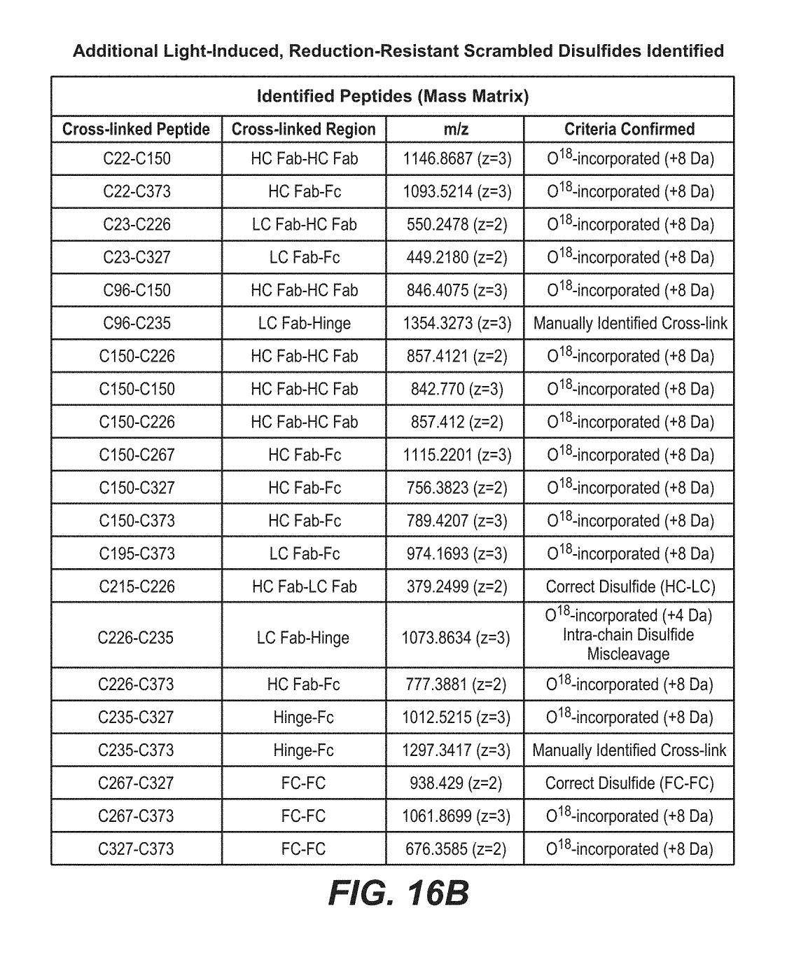

The concept for O¹⁸-labeling follows the sequence of analysis: 1) Tryptic digestion in the presence of H₂O¹⁶ and H₂O¹⁸; 2) Identification of putative dipeptides by the incorporation of four O¹⁸ molecules to the c-terminus tryptic carboxylic acids (+8 Da mass shift compared to H₂O¹⁶ digested sample); 3) In silico fragment mass database search to identify partial peptide sequences based on protease specific constraints; 4) Extension to full putative peptides; 5) Deduction of cross-linking chemistry and residues involved. Using this methodology, the high mass accuracy peptide fragments for a cross-link parent ion with a molecular weight Molecular Mass=3889.0041 Da, were analyzed in silico and a latter of the putative peptides was observed and the identified cross-linked peptide between hinge (C232) and Fc (C373) was confirmed (FIGS. 15A-1 and 15A-2). Using the same methodology, the high mass accuracy peptide fragments for a second cross-link parent ion with a molecular weight Molecular Mass=4059.9645 Da, were analyzed in silico, and a latter of the putative peptides was observed. The cross-linked peptide between hinge and Fab was identified and the cross-linked site was confirmed to be between hinge (C235) and Fab (C96) (FIGS. 15B-1 and 15B-2). Additional RR cross-linked Cys residues were identified and confirmed by LC/MS peptide mapping (FIG. 16B).

### Example 5 Activity Assays of Variants

The biological activity and potency of the variant described herein are analyzed. The binding and neutralizing activities of the variants are tested by the assays described throughout the disclosure and known in the art. For example, binding affinity of the variants to IL-17A homodimer, IL-17F homodimer and/or IL-17AF heterodimer can be determined by the ELISA assay described above or BIACORE™ assay as described in, e.g., U.S. Pat. No. 8,715,669 and U.S. Pat. No. 8,790,642 (incorporated herein by reference in their entireties for any purposes). Briefly, binding affinities of an anti-IL-17 A/F antibody variant can be measured by Surface Plasmon Resonance (SRP) using a BIAcore™-3000 instrument. The antibody is captured by mouse anti-human Fc antibody (GE Healthcare, cat# BR-1008-39) coated on CMS biosensor chips to achieve approximately 200 response units (RU). For kinetics measurements, two-fold serial dilutions (0.98 nM to 125 nM) of human IL-17A, IL-17 F or IL-17A/F, can be injected in PBT buffer (PBS with 0.05% Tween 20) at 25° C. with a flow rate of 30 μl/min. The cytokines are available through commercial sources such as R&D Systems. Association rates (kₒₙ) and dissociation rates (k_{off}) are calculated using a simple one-to-one Langmuir binding model (BIAcore Evaluation Software version 3.2). The equilibrium dissociation constant (K_{D}) is calculated as the ratio k_{off}/kₒₙ. The isolated and/or enriched variants described herein show reduced binding affinity as compared to the main species of anti-IL-17A/F antibody or a composition comprising predominantly the main species of anti-IL17A/F antibody.

Neutralizing activity of the variants can be determined by evaluating the inhibition of IL-17A or F-induced cytokine induction, for example, IL6 or G-CSF. For example, human neonatal foreskin fibroblasts (Invitrogen) are seeded in 96-well plate at 2×10⁴ cells/150 μl media/well on day 1. Media is replaced with cytokine/antibody containing media (150 μl) on day 2. Suitable amount of recombinant human IL-17A homodimer (e.g., at 5 ng/ml), IL-17F homodimer (e.g., 50 ng/ml) and IL-17AF heterodimer (e.g., 25 ng/ml) can be used. Supernatant is harvested 24 hours later and G-CSF ELISA was performed to measure G-CSF induction. Data are plotted in PRISM and IC50/90 values calculated using the same software. The one or more glycosylation variant, acidic variant, HMWS variant and RR-cross linked variant show reduced neutralizing activity as compared to the main species of anti-IL17A/F antibody.

It should be understood that the foregoing disclosure emphasizes certain specific embodiments of the invention and that all modifications or alternatives equivalent thereto are within the spirit and scope of the invention as set forth in the appended claims.

1. An isolated composition comprising an anti-IL-17A and anti-IL-17 F cross-reactive antibody comprising a heavy chain variable region CDR1 comprising the amino acid sequence of SEQ ID NO:1, CDR2 comprising the amino acid sequence of SEQ ID NO:2, CDR3 comprising the amino acid sequence of SEQ ID NO:3, and a light chain variable region CDR1 comprising the amino acid sequence of SEQ ID NO:4, CDR2 comprising the amino acid sequence of SEQ ID NO:5, and CDR3 comprising the amino acid sequence of SEQ ID NO:6, and a glycosylation variant thereof.

2. The composition of claim 1, wherein the glycosylation is in the heavy chain variable region.

3. The composition of claim 1, wherein the glycosylation variant is a heterodimer variant in which only one heavy chain variable region is glycosylated.

4. The composition of claim 1, wherein the glycosylation variant is a homodimer variant in which both heavy chain variant regions are glycosylated.

5. The composition of claim 1, wherein the glycosylation is in the heavy chain variable region CDR2.

6. The composition of claim 5, wherein the glycosylation site is at the Asn of SEQ ID NO:2.

7. The composition of claim 1, wherein the amount of the glycosylation variant in the composition is no more than about 4%.

8. The composition of claim 1, wherein the amount of the glycosylation variant in the composition is no more than about 2%.

9. The composition of claim 1, wherein the heavy chain variable region comprises the amino acid sequence of SEQ ID NO:7

10. The composition of claim 1, wherein the light chain variable region comprises the amino acid sequence of SEQ ID NO:8.

11. The composition of claim 1, wherein the antibody comprises a heavy chain comprising the amino acid sequence of SEQ ID NO:9.

12. The composition of claim 11, wherein the antibody comprises a light chain comprising the amino acid sequence of SEQ ID NO:10.

13. (canceled)

14. The composition of claim 1, wherein the amount of the glycosylation variant in the composition is no more than about 4% as measured by size exclusion high performance liquid chromatography (SE-HPLC).

15. The composition of claim 14, wherein the amount of the glycosylation variant in the composition is no more than about 2% as measured by SE-HPLC.

16. The composition of claim 1, wherein the composition further comprises one or more additional variants of the antibody, wherein the additional one or more variants are selected from the group consisting of a high-molecular-weight-species (HMWS) variant, a reduction-resistant (RR) cross-linked variant, and an acidic variant.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. An isolated composition comprising an anti-IL-17A and anti-IL-17 F cross reactive antibody comprising a heavy chain variable region CDR1 having the amino acid sequence of SEQ ID NO: 1, CDR2 having the amino acid sequence of SEQ ID NO:2, CDR3 having the amino acid sequence of SEQ ID NO:3, and a light chain variable region CDR1 having the amino acid sequence of SEQ ID NO:4, CDR2 having the amino acid sequence of SEQ ID NO:5, and CDR3 having the amino acid sequence of SEQ ID NO:6, wherein the composition comprises one or more of a glycosylation variant, a reduction-resistant (RR) cross-linked variant, a high-molecular-weight-species (HMWS) variant, or an acidic variant.

35. The composition of claim 34, wherein the composition comprises an RR cross-linked variant.

36. The composition of claim 35, wherein the amount of the RR cross-linked variant in the composition is no more than about 3% as determined by organic phase size exclusion chromatography (OP-SEC).

37. The composition of claim 34, wherein the composition comprises a HMWS variant.

38. The composition of claim 37, wherein the amount of the HMWS variant in the composition is no more than about 1% as determined by SE-HPLC.

39. The composition of claim 34, wherein the composition comprises an acidic variant.

40. The composition of claim 39, wherein the amount of the acidic variant in the composition is no more than about 42% as determined by imaged capillary isoelectric-focusing (icIEF).

41. The composition of claim 34, wherein the composition comprises a HMWS variant, an RR cross-linked variant and an acidic variant.

42. The composition of claim 41, wherein the amount of the HMWS variant in the composition is no more than about 1% as determined by SE-HPLC, the amount of the acidic variant in the composition is no more than about 42% as determined by icIEF, and the amount of the RR cross-linked variant in the composition is no more than about 3% as determined by OP-SEC.

43. The composition of claim 42, further comprising a glycosylation variant.

44. The composition of claim 43, wherein the amount of the glycosylation variant in the composition is no more than about 2% as determined by SE-HPLC.

45. A pharmaceutical composition comprising the composition of claim 1 and at least pharmaceutically acceptable excipient.

46. An article of manufacture comprising a container with the pharmaceutical composition of claim 45 and a package insert with prescribing information instructing the use thereof to use the pharmaceutical composition to treat a patient in need thereof.

47. A method of treating an immune-related disease, an inflammatory disease or a cell proliferation-related disease comprising administering to a subject in need thereof the pharmaceutical composition of claim 45.

48. The method of claim 47, wherein the immune-related disease is asthma, multiple sclerosis, rheumatoid arthritis, inflammatory bowel disease, ulcerative colitis, lupus erythematosus, psoriasis, chronic obstructive pulmonary disease, idiopathic pulmonary fibrosis.

49. The method of claim 47, wherein the cell proliferation-related disease is cancer.

50. A pharmaceutical composition comprising the composition of claim 34 and at least pharmaceutically acceptable excipient.

51. An article of manufacture comprising a container with the pharmaceutical composition of claim 50 and a package insert with prescribing information instructing the use thereof to use the pharmaceutical composition to treat a patient in need thereof.

52. A method of treating an immune-related disease, an inflammatory disease or a cell proliferation-related disease comprising administering to a subject in need thereof the pharmaceutical composition of claim 50.

53. The method of claim 52, wherein the immune-related disease is asthma, multiple sclerosis, rheumatoid arthritis, inflammatory bowel disease, ulcerative colitis, lupus erythematosus, psoriasis, chronic obstructive pulmonary disease, idiopathic pulmonary fibrosis.

54. The method of claim 52, wherein the cell proliferation-related disease is cancer.


SEQUENCE LISTING (16 sequences)

SEQ ID NO: 1 - protein, 5 aa, Homo sapiens
DYAMH

SEQ ID NO: 2 - protein, 17 aa, Homo sapiens
GINWSSGGIGYADSVKG

SEQ ID NO: 3 - protein, 14 aa, Homo sapiens
DIGGFGEFYWNFGL

SEQ ID NO: 4 - protein, 11 aa, Homo sapiens
RASQSVRSYLA

SEQ ID NO: 5 - protein, 7 aa, Homo sapiens
DASNRAT

SEQ ID NO: 6 - protein, 10 aa, Homo sapiens
QQRSNWPPAT

SEQ ID NO: 7 - protein, 123 aa, Artificial Sequence
  source: /note=“Description of Artificial Sequence Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSS

SEQ ID NO: 8 - protein, 108 aa, Artificial Sequence
  source: /note=“Description of Artificial Sequence Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIK

SEQ ID NO: 9 - protein, 452 aa, Artificial Sequence
  source: /note=“Description of Artificial Sequence Synthetic polypeptide”
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 215 aa, Artificial Sequence
  source: /note=“Description of Artificial Sequence Synthetic polypeptide”
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 22 aa, Artificial Sequence
  source: /note=“Description of Artificial Sequence Synthetic peptide”
GLEWVSGINWSSGGIGYADSVK

SEQ ID NO: 12 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 13 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 14 - protein, 10 aa, Artificial Sequence
  source: /note=“Description of Artificial Sequence Synthetic peptide”
NQVSLTCLVK

SEQ ID NO: 15 - protein, 26 aa, Artificial Sequence
  source: /note=“Description of Artificial Sequence Synthetic peptide”
THTCPPCPAPELLGGPSVFLFPPKPK

SEQ ID NO: 16 - protein, 11 aa, Artificial Sequence
  source: /note=“Description of Artificial Sequence Synthetic peptide”
AEDTALYYCAR